(19) United States
(12) Patent Application Publication
(10) Pub. No.: US 2023/0235050 A1
(43) Pub. Date: Jul. 27, 2023

(54) SINGLE-DOMAIN ANTIBODIES DIRECTED AGAINST LILRB2

(71) Applicant: Invectys SAS, Paris (FR)

(72) Inventors: Maria LOUSTAU, Paris (FR); Lise HUNAULT, Voisins-Le-Bretonneux (FR); Pierre LANGLADE DEMOYEN, Neuilly-sur-Seine (FR); Julien CAUMARTIN, Le Vesinet (FR)

(73) Assignee: Invectys SAS, Paris (FR)

(21) Appl. No.: 17/762,270

(22) PCT Filed: Sep. 18, 2020
(86) PCT No.: PCT/EP2020/076198

§ 371 Date: Mar. 21, 2022

(30) Foreign Application Priority Data
Sep. 20, 2019 (EP) 19306148.8

(51) Int. Cl. C07K 16/28; C07K 14/725; C12N 15/85; G01N 33/574

(52) U.S. Cl. CPC C07K 16/2803; C07K 14/7051; C12N 15/85; G01N 33/57484; A61K 2039/505

(57) ABSTRACT

The present invention relates to single-domain antibodies (sdAbs) directed against Leukocyte immunoglobulin-like receptor subfamily B member 2 (LILRB2), pharmaceutical compositions comprising

### CROSS-REFERENCE TO RELATED APPLICATIONS

This application is a 35 U.S.C. § 371 national phase application of International Patent Application No. PCT/EP2020/076198, filed Sep. 18, 2020, which claims the benefit of European Patent Application No. 19306148.8, filed Sep. 20, 2019, each of which is incorporated by reference herein in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: INVE-011_01US_SubSeqList.txt, date recorded: Aug. 19, 2022, file size ˜27,201 bytes).

### FIELD OF THE INVENTION

The invention pertains in the field of immunotherapy and immunodiagnostic. The present invention provides single-domain antibodies (sdAbs) directed against Leukocyte immunoglobulin-like receptor subfamily B member 2 (LILRB2).

### TECHNOLOGICAL BACKGROUND

Leukocyte Immunoglobulin (Ig)-like receptors (LILBRs) are inhibitory receptors for which the cytoplasmic tail is composed of ITIMs (Immunoreceptor tyrosine-based inhibitory motifs). Whereas LILRB1 is expressed on all immune cell subsets, LILRB2 expression is limited to antigen presenting cells (APCs) such as monocytes, dendritic cells and macrophages.

LILRB2 interacts with CD1d, several molecules from the complement cascade, (C4d, C3d, C4b, C3b and iC3b), angiopoietin-like 2 and 5 (ANGPTL2/5) proteins, B-amyloid 1-42 and myelin-derived inhibitors (Nogo66, MAG) and either with classical (HLA-A, -B and -C) or non-classical MHC class I molecules (HLA-E, F and G). It was particularly demonstrated that interaction between LILRB2 and HLA-G, expressed on immune cells, inhibits cell functions and can induce immunosuppressive cells. Indeed, the interaction between HLA-G and LILRB2 present in dendritic cells (DCs) inhibits their maturation and renders them tolerogenic.

Interestingly, LILRB2 receptor was shown to be expressed in several types of cancer and frequently associated with metastasis. Although LILRB2 is an inhibitory receptor, its expression by tumors was shown to increase tumor cell proliferation and motility. Indeed, upon binding to HLA-G or ANGPTL2, LILRB2 receptor inhibits pathways that repress proliferation, growth and dissemination of tumor cells. Noteworthy, LILRB2 is expressed by tumor-associated macrophages (TAM), especially in the context of solid tumors. These macrophages display a M2-phenotype which is associated with the inhibition of immune cell infiltration and functions that favors the proliferation of cancer cells. Since LILRB2 receptor expression is restricted to APCs in healthy individuals, its neo-expression in tumors and its strong upregulation by tolerogenic DCs and TAMs makes of LILRB2 receptor an excellent tumor associated antigen (TAA) to target for immunotherapeutic treatments.

However, to date, there is no efficient immunotherapeutic agent that is capable of blocking LILRB2. The generation of blocking anti-LILRB2 monoclonal antibodies (mAb) would pave the way to new immunotherapeutic treatments. However, the large size of mAb (˜150 kDa) is a main drawback since it dampens their tumor penetration and therefore limits their application for solid cancers, which are still the most difficult cancers to treat. There remains therefore a significant need in the art for new and improved agents to target such cancers.

Camelidae members naturally produce different class of antibodies: (i) the conventional heavy-chain antibodies containing two light and two heavy chains (˜150 kDa), (ii) homodimeric heavy-chain antibodies comprising only H chains (HcAbs; ˜95 kDa) and (iii) additional IgG isotypes based on a unique heavy chain. These heavy-chain-only antibodies have demonstrated to have high binding affinity and specificity for their antigen, similarly to conventional mAbs.

The variable domain of the heavy chain from HcAbs (i.e single domain antibodies (sdAbs) or Nanobodies© (Nbs)) is responsible for the antigen binding and specificity and can be isolated from HcAbs without the loss of their binding properties. Their small size, generally around 15-20 kDa, is an important advantage when targeting solid tumors. In fact, they should be able to penetrate the fibrous microenvironment surrounding cancer cells with more efficiency, and reach target cells such as macrophages settled in this stroma. Then, sdAb are excellent candidates in the context of targeting LILRB2 receptors displayed on solid tumors and on TAMs.

The inventors have now made a significant technical contribution to the art in developing anti-LILRB2 single domain antibodies (sdAbs).

### SUMMARY OF THE INVENTION

The invention concerns a single domain antibody (sdAb) which specifically binds to or specifically recognizes Leukocyte immunoglobulin-like receptor subfamily B member 2 (LILRB2), preferably human LILRB2.

Preferably, said sdAb anti-LILRB2 does not bind Leukocyte immunoglobulin-like receptor subfamily B member 1 (LILRB1), preferably human LILRB1.

In one aspect, the sdAb according to the invention comprises at least one complementarity determining regions (CDR) which comprises or consists in the sequence set forth in SEQ ID NO: 3, 6, 9, 12, 15, 18, 21, 24, 27, 30 or 33 or comprises, or consists in an amino acid sequence which differs from SEQ ID NO: 3, 6, 9, 12, 15, 18, 21, 24, 27, 30 or 33 in virtue of one, two, or three amino acid modifications.

Preferably, the sdAb according to the invention comprises three CDRs, wherein:
(a) CDR1 comprises, or is of, SEQ ID NO:1 or has an amino acid sequence which differs from SEQ ID NO:1 in virtue of one, two, or three amino acid modifications, and
   CDR2 comprises, or is of, SEQ ID NO:2 or has an amino acid sequence which differs from SEQ ID NO:2 in virtue of one, two, or three amino acid modifications, and
   CDR3 comprises, or is of, SEQ ID NO:3 or has an amino acid sequence which differs from SEQ ID NO:3 in virtue of one, two, three or four amino acid modifications; or
(b) CDR1 comprises, or is of, SEQ ID NO:4 or has an amino acid sequence which differs from SEQ ID NO:4 in virtue of one, two, or three amino acid modifications, and
   CDR2 comprises, or is of, SEQ ID NO:5 or has an amino acid sequence which differs from SEQ ID NO:5 in virtue of one, two, or three amino acid modifications, and
   CDR3 comprises, or is of, SEQ ID NO:6 or has an amino acid sequence which differs from SEQ ID NO:6 in virtue of one, two, three or four amino acid modifications; or
(c) CDR1 comprises, or is of, SEQ ID NO:7 or has an amino acid sequence which differs from SEQ ID NO:7 in virtue of one, two, or three amino acid modifications, and
   CDR2 comprises, or is of, SEQ ID NO:8 or has an amino acid sequence which differs from SEQ ID NO:8 in virtue of one, two, or three amino acid modifications, and
   CDR3 comprises, or is of, SEQ ID NO:9 or has an amino acid sequence which differs from SEQ ID NO:9 in virtue of one, two, three or four amino acid modifications; or
(d) CDR1 comprises, or is of, SEQ ID NO:10 or has an amino acid sequence which differs from SEQ ID NO:10 in virtue of one, two, or three amino acid modifications, and
   CDR2 comprises, or is of, SEQ ID NO:11 or has an amino acid sequence which differs from SEQ ID NO:11 in virtue of one, two, or three amino acid modifications, and
   CDR3 comprises, or is of, SEQ ID NO:12 or has an amino acid sequence which differs from SEQ ID NO:12 in virtue of one, two, three or four amino acid modifications; or
(e) CDR1 comprises, or is of, SEQ ID NO:13 or has an amino acid sequence which differs from SEQ ID NO:13 in virtue of one, two, or three amino acid modifications, and
   CDR2 comprises, or is of, SEQ ID NO:14 or has an amino acid sequence which differs from SEQ ID NO:14 in virtue of one, two, or three amino acid modifications, and
   CDR3 comprises, or is of, SEQ ID NO:15 or has an amino acid sequence which differs from SEQ ID NO:15 in virtue of one, two, three or four amino acid modifications; or
(f) CDR1 comprises, or is of, SEQ ID NO:16 or has an amino acid sequence which differs from SEQ ID NO:16 in virtue of one, two, or three amino acid modifications, and
   CDR2 comprises, or is of, SEQ ID NO:17 or has an amino acid sequence which differs from SEQ ID NO:17 in virtue of one, two, or three amino acid modifications, and
   CDR3 comprises, or is of, SEQ ID NO:18 or has an amino acid sequence which differs from SEQ ID NO:18 in virtue of one, two, three or four amino acid modifications; or
(g) CDR1 comprises, or is of, SEQ ID NO:19 or has an amino acid sequence which differs from SEQ ID NO:19 in virtue of one, two, or three amino acid modifications, and
   CDR2 comprises, or is of, SEQ ID NO:20 or has an amino acid sequence which differs from SEQ ID NO:20 in virtue of one, two, or three amino acid modifications, and
   CDR3 comprises, or is of, SEQ ID NO:21 or has an amino acid sequence which differs from SEQ ID NO:21 in virtue of one, two, three or four amino acid modifications; or
(h) CDR1 comprises, or is of, SEQ ID NO:22 or has an amino acid sequence which differs from SEQ ID NO:22 in virtue of one, two, or three amino acid modifications, and
   CDR2 comprises, or is of, SEQ ID NO:23 or has an amino acid sequence which differs from SEQ ID NO:23 in virtue of one, two, or three amino acid modifications, and
   CDR3 comprises, or is of, SEQ ID NO:24 or has an amino acid sequence which differs from SEQ ID NO:24 in virtue of one, two, three or four amino acid modifications; or
(i) CDR1 comprises, or is of, SEQ ID NO:25 or has an amino acid sequence which differs from SEQ ID NO:25 in virtue of one, two, or three amino acid modifications, and
   CDR2 comprises, or is of, SEQ ID NO:26 or has an amino acid sequence which differs from SEQ ID NO:26 in virtue of one, two, or three amino acid modifications, and
   CDR3 comprises, or is of, SEQ ID NO:27 or has an amino acid sequence which differs from SEQ ID NO:27 in virtue of one, two, three or four amino acid modifications; or
(j) CDR1 comprises, or is of, SEQ ID NO:28 or has an amino acid sequence which differs from SEQ ID NO:28 in virtue of one, two, or three amino acid modifications, and
   CDR2 comprises, or is of, SEQ ID NO:29 or has an amino acid sequence which differs from SEQ ID NO:29 in virtue of one, two, or three amino acid modifications, and
   CDR3 comprises, or is of, SEQ ID NO:30 or has an amino acid sequence which differs from SEQ ID NO:30 in virtue of one, two, three or four amino acid modifications; or
(k) CDR1 comprises, or is of, SEQ ID NO:31 or has an amino acid sequence which differs from SEQ ID NO:31 in virtue of one, two, or three amino acid modifications, and
   CDR2 comprises, or is of, SEQ ID NO:32 or has an amino acid sequence which differs from SEQ ID NO: 32 in virtue of one, two, or three amino acid modifications, and
   CDR3 comprises, or is of, SEQ ID NO:33 or has an amino acid sequence which differs from SEQ ID NO:33 in virtue of one, two, three or four amino acid modifications.

Particularly, the anti-LILRB2 sdAb comprises three CDRs, wherein CDR1 comprises, or is of, SEQ ID NO:1, and CDR2 comprises, or is of, SEQ ID NO:2, and CDR3 comprises, or is of, SEQ ID NO:3.

In a particular aspect, the sdAb anti-LILRB2 comprises or consists in a sequence defined in any of the sequence SEQ ID No: 34 to SEQ ID No: 44 or a sequence having at least 80% sequence identity thereto, preferably at least 90%, 92%, 94%, 95%, 96%, 97%, 98%, 99% or more amino-acid sequence identity thereto.

Particularly, the anti-LILRB2 sdAb comprises or consists in a sequence defined in SEQ ID No: 34.

Preferably, the sdAb anti-LILRB2 according to the invention inhibits the interaction between LILRB2 and human leukocyte antigen-G (HLA-G) and/or the interaction between LILRB2 and Angiopoietin Like 2 (ANGPTL2).

In another aspect, the invention concerns an isolated nucleic acid comprising a sequence encoding a sdAb anti-LILRB2 according to the invention, preferably defined by a sequence selected in the group consisting of SEQ ID: 45-55.

The invention also relates to a vector comprising the isolated nucleic acid according to the invention, but also to a chimeric antigen receptor (CAR) comprising the sdAb or the isolated nucleic acid according to the invention.

In a particular aspect, the invention concerns a cell comprising the isolated nucleic acid or the vector according to the invention or expressing the CAR disclosed herein. Preferably, the cell is selected from a group consisting of a T cell, CD4⁺ T cell, CD8⁺ T cell, B cell, NK cell, NKT cell, monocyte and dendritic cell, preferably the cell being a T cell, a B cell or a NK cell.

The invention further relates to a pharmaceutical composition comprising a sdAb, the isolated nucleic acid, the vector, the CAR or the cell expressing a CAR according to the invention, and optionally a pharmaceutically acceptable carrier.

In one aspect, the sdAb, the isolated nucleic acid, the vector, the CAR, the cell or the pharmaceutical composition according to the invention is for use in the treatment of cancer, preferably wherein the cancer overexpresses LILBR2 more preferably a cancer selected from the group consisting of lung cancer, non-small cell lung cancer (NSCLC), pancreatic cancer, pancreatic ductal carcinoma, Chronic Lymphocytic Leukemia (CLL), Acute Myeloid Leukemia (AML), endometrial cancer, hepatocellular carcinoma, melanoma, ovarian cancer, breast cancer, colorectal cancer, glioma, stomach cancer, renal cancer, testis cancer, Esophageal cancer, Cervical cancer, Lewis Lung cancer of mice, Leukemia, Thyroid cancer, Liver cancer, Urothelial cancer and Head and neck cancer.

The invention finally relates to the use of the sdAb anti-LILRB2 according to the invention, for detecting LILRB2 on tumoral cells or tissues in vitro or ex vivo.

### FIGURES

FIG. 1. Alpaca immunization and VHH specificity identification. A) Alpaca immunization protocol with rhLILRB2-Fc proteins. B) Serum from immunized alpaca was tested in ELISA with different dilutions. C) Selection of VHHs was done using phage-display vectors and biopanning technique and assessed against rhLILRB2-Fc. Positive anti-LILRB2 VHHs are circled in full line while negative are in dotted line.

FIG. 2. B8, C7 and C9 Nbs recognize linear epitopes of rhLILRB2. Denaturated rhLILRB2-Fc (rhILT4-Fc), rhLILRB2 (rhILT4) and rhLILRB1 (rhILT2) proteins were transferred onto membranes by Western blotting. A) rhLILRB2-Fc, rhLILRB2 and rhLILRB1 proteins were incubated with control anti-LILRB2 Abs (H-300 and 42D1), control anti-LILRB1 (GHI/75 and HP-F1). B) rhLILRB2-Fc, rhLILRB2 and rhLILRB1 proteins were incubated with B8, C7 and C9 Nbs. Ab binding was detected using HRP-labeled goat-anti-rat antibodies for H-300 and HRP-labeled goat-anti-mouse Abs for 42D1, GHI/75 and HP-F1 and HRP labeled mouse anti-c-Myc tag the Nbs.

FIG. 3. Binding of Nbs (B8, C7, C9) to rhILT4 denatured (D1-D4 domains) and absence or binding of Nbs (B8, C7, C9) to rhILT2 denatured (D1-D4).

FIG. 4. Nbs specificity for LILRB2 transduced D1.1 cell line. LILRB2 D1.1 cell line was co-incubated with 42D1 control antibody or anti-LILRB2 Nbs and analyzed by flow cytometry.

FIG. 5. Nbs specificity for LILRB2 receptors on monocytes from PBMCs. Monocytes were isolated from PBMCs and then stained for LILRB2 receptors expression with the 42D1 control antibody and Nbs in comparison with an irrelevant control Nb and analyzed by flow cytometry. Monocytes were identified from other leukocytes with anti-CD14 and anti-LILRB1 Abs.

FIG. 6. Blocking capacity of anti-LILRB2 Nbs against LILRB2/HLA-G6 interaction. As depicted in the study design panel (upper right), microtiter plates were coated with rhLILRB2-Fc protein before being co-incubated with individual Nbs. HLA-G6 V5 tagged protein was then added and detection of HLA-G6-V5 protein was performed using HRP conjugated anti-V5 Ab. Values were normalized to the mean absorbance intensity of the negative control (rhLILRB2-Fc incubated only with HLA-G6-V5 protein in absence of Nb or control Ab) (n=3).

FIG. 7. Blocking capacity of anti-LILRB2 Nbs against LILRB2/ANGPTL2 interaction. As depicted in the study design panel (upper right), microtiter plates were coated with rhLILRB2-Fc protein before being co-incubated with individual Nbs. ANGPTL2 protein was then added and detection of ANGPTL2 was performed using an anti-ANGPTL2 purified Ab followed by HRP conjugated anti-rabbit Ab. Values were normalized to the mean absorbance intensity (MFI) of the negative control (rhLILRB2-Fc incubated only with ANGPLT2 protein in absence of Nb or control Ab (n=1).

### DETAILED DESCRIPTION OF THE INVENTION

### Definitions

As used herein, “Leukocyte immunoglobulin-like receptor subfamily B member 2” or “LILRB2” refers to a member of the leukocyte immunoglobulin-like receptor (LIR) family, particularly to the subfamily B class of LIR receptors which contain two or four extracellular immunoglobulin domains, a transmembrane domain, and two to four cytoplasmic immunoreceptor tyrosine-based inhibitory motifs (ITIMs). LILRB2 is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. LILRB2 has known alternative names such as LIR2, CD85 antigen-like family member D, CD85D, Immunoglobulin-like transcript 4, ILT4, Monocyte/macrophage immunoglobulin-like receptor 10 or MIR-10. In the context of the invention, this term particularly refers to human LILRB2. Human LILRB2 is known in the art for example under the UniProt accession number Q8N423. For example, the human LILRB2 amino acid sequence is about 598 amino acids, the gene being located in cluster at chromosomal region 19q13.4. Human LILRB2 has four known isoforms produced by alternative splicing. Isoform 1 has been chosen as the canonical sequence and is described under the accession number Uniprot Q8N423-1, isoform 2 differs from isoform 1 by the deletion of amino-acid at position 437 and is described under the accession number Uniprot Q8N423-2, isoform 3 differs from isoform 1 by the deletion of amino-acid at positions 495-510 and 511-598 and is described under the accession number Uniprot Q8N423-3, isoform 4 differs from isoform 1 by the deletion of amino-acids at position 1-116 and is described under the accession number Uniprot Q8N423-4. In the context of the invention, the term “LILRB2” encompasses all isoforms of LILRB2.

As used herein, “heavy-chain antibodies” (HCAb) refer to immunoglobulins which are devoid of light chains and consist in two heavy chains. Each heavy chain comprises a constant region (CH) and a variable domain (VH) which enables the binding to a specific antigen, epitope or ligand. As used herein, HCAbs encompass heavy chain antibodies of the camelid-type in which each heavy chain comprises a variable domain called VHH and two constant domains (CH2 and CH3). Noteworthy, camelid HCAbs lack the first constant domain (CH1). Such heavy-chain antibodies directed against a specific antigen can be obtained from immunized camelids. As used herein, “camelids” encompass dromedary, camel, llama and alpaca. Camelid HCAbs have been described by Hamers-Casterman et al., Nature, 1993, 363:446. Other examples of HCAb are immunoglobulin-like structures from cartilaginous fishes (Ig-NAR) such as nurse shark (Ginglymostoma cirratum) and wobbegong shark (Orectolobus maculates).

As used herein, a “single-domain antibody” (sdAb or Nb) refers to a single-variable domain, derived from a heavy-chain only antibody, which is able to bind an antigen, an epitope or a ligand alone, that is to say, without the requirement of another binding domain. A single domain antibody may derive from, or consists in, a VHH or a V-NAR. VHH refers to the variable domain found in HCAb of Camelidae. V-NAR refers to the variable domain found in immunoglobulin-like structures (Ig-NAR) discovered in cartilaginous fishes. As an alternative, single-domain antibody may be obtained from naïve synthetic libraries. For review about single-domain antibodies, one may refer to Saerens et al., Current Opinion in Pharmacology, 2008, 8:600-608, Muyldermans et al., Vet Immunol Immunopathol. 2009 Mar. 15; 128(1-3):178-83, and/or Muyldermans 2013, Annu Rev Biochem. 2013; 82:775-97, the disclosure of which being incorporated by reference.

As used herein, “bind” or “binding” refer to peptides, polypeptides, proteins, fusion proteins and antibodies (including sdAb) that recognize and contact an antigen. By “specifically bind” or “immunospecifically bind”, it is meant that the antibody recognizes a specific antigen, but does not substantially recognize nor bind other molecules or antigens in a sample. In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope). As used herein, the term “specific binding” means the contact between an antibody and an antigen with a binding affinity of at least 10⁻⁶ or 10⁻⁷ M. In certain aspects, antibodies bind with affinities of at least about 10⁻⁸ M, and preferably 10⁻⁹ M, 10⁻¹⁰ M, 10⁻¹¹ M, 10⁻¹² M.

The terms “sdAb that specifically binds to LILRB2” and analogous terms, as used herein, refer to sdAbs that specifically recognize LILRB2 and do not or weakly recognize other antigens (including other members of the LILR family, for example such as LILRB1). Preferably, sdAbs that specifically bind to LILRB2 have a higher affinity to this antigen when compared to the affinity to other antigens or fragments thereof, including other LILR family members, for example such as LILRB1, preferably by at least a factor 10, 100 or 1000.

The affinity of an antibody or an sdAb can be a measure of its binding with a specific antigen at a single antigen-antibody site and is in essence the summation of all the attractive and repulsive forces present in the interaction between the antigen-binding site of an antibody and a particular epitope. The affinity of an antibody or a sdAb to a particular antigen (e.g. LILRB2) may be expressed by the equilibrium constant K of dissociation, defined by the equation Kd=[Ag][Ab]/[Ag Ab], which represents the affinity of the antibody-combining site; where [Ag] is the concentration of free antigen (M), [Ab] is the concentration of free antibody (M) and [Ag Ab] is the concentration (M) of the antigen-antibody complex. Where the antigen and antibody or sdAb react strongly together there will be very little free antigen or free antibody or sdAb, and hence the equilibrium constant or affinity of the antibody or a sdAb will be low.

The “identity” of the “percentage identity” between two amino acid sequences (A) and (B) is determined by comparing the two sequences aligned in an optimal manner, through a window of comparison. Said alignment of sequences can be carried out by well-known methods, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on:

http://www.ebi.ac.uk/Tools/services/web/toolform.ebi?tool=emboss_needle&context=protein, using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

As used herein, by “amino acid modification” is meant a change in the amino acid sequence of a polypeptide. “Amino acid modifications” which may be also termed “amino acid changes”, herein include amino acid mutations such as substitution, insertion, and/or deletion in a polypeptide sequence. By “amino acid substitution” or “substitution” herein is meant the replacement of an amino acid at a particular position in a parent polypeptide sequence with another amino acid. Preferably, substitutions are silent substitutions. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art.

As used herein, “parent polypeptide” or “polypeptide parent” refer to an unmodified polypeptide that is subsequently modified to generate a variant. In the context of the invention, the parent polypeptide may be a VHH from a naturally-occurring HCAb.

“Variant polypeptide”, “polypeptide variant” or “variant”, as used herein, refers to a polypeptide sequence that differs from that of a parent polypeptide sequence by virtue of at least one amino acid modification. For instance, in the context of the invention, a variant may be a variant of a VHH from a naturally-occurring HCAb. Typically, a variant comprises from 1 to 50 amino acid modifications, preferably from 1 to 40 amino acid modifications. In particular, the variant may have from 1 to 30 amino acid changes, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid changes as compared to its parent. The variants may comprise one or several amino acid substitutions, and/or, one or several amino acid insertions, and/or one or several amino acid deletions. In some embodiments, the variant may comprise one or several conservative substitutions, e.g. as shown hereabove. In some further embodiments, the variant of a sdAb may comprise one or several amino acid modifications in the CDR domains of the parent sdAb. As CDR3 is commonly used to define sdAb families having the same recognition pattern, such modifications in CDR3 may lead to a new sdAb family having distinct binding properties (for instance an increased binding property) as compared to the parent sdAb whereas modifications in CDR1 or CDR2 may lead to define different members of the same family (i.e. having the same CDR3 but different CDR1 and/or CDR2). In some other embodiments, the variant of the parent sdAb may comprise one or several amino acid modifications in at least one framework domain.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect.

As used herein, the term “disorder” or “disease” refers to the incorrectly functioning organ, part, structure, or system of the body resulting from the effect of genetic or developmental errors, infection, poisons, nutritional deficiency or imbalance, toxicity, or unfavourable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

The term “cancer” as used herein is defined as disease characterized by the rapid and uncontrolled growth of aberrant cells. Cancer cells can spread locally or through the bloodstream and lymphatic system to other parts of the body, for example in metastasis.

As used herein, the term “subject”, “host”, “individual,” or “patient” refers to human and veterinary subjects particularly to an animal, preferably to a mammal, even more preferably to a human, including adult and child. However, the term “subject” also encompasses non-human animals, in particular mammals such as dogs, cats, horses, cows, pigs, sheep and non-human primates, among others.

As used herein, a “pharmaceutical composition” refers to a preparation of one or more of the active agents, such as comprising an antigen binding domain of an anti-LILRB2 antibody or sdAb according to the invention, with optional other chemical components such as physiologically suitable carriers and excipients. The purpose of a pharmaceutical or veterinary composition is to facilitate administration of the active agent to an organism. Compositions of the present invention can be in a form suitable for any conventional route of administration or use. In one embodiment, a “pharmaceutical composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers.

An “acceptable vehicle” or “acceptable carrier” as referred to herein, is any known compound or combination of compounds that are known to those skilled in the art to be useful in formulating pharmaceutical or veterinary compositions.

A “therapeutically effective amount” is an amount which, when administered to a subject, is the amount of active agent that is needed to treat the targeted disease or disorder, or to produce the desired effect.

The “effective amount” will vary depending on the agent(s), the disease and its severity and the age, weight, and characteristics of the subject to be treated.

As used herein, the term “medicament” refers to any substance or composition with curative or preventive properties against disorders and/or diseases.

Single Domain Antibodies Directed Against LILRB2

As mentioned above, sdAb molecules correspond to the variable region of heavy chain only antibodies that are naturally devoid of light chains. The antigen-binding surfaces of sdAbs are usually more convex (or protruding) than those of conventional antibodies, which are usually flat or concave.

A single-domain antibody according to the invention comprises a single variable domain derived from an antibody able to bind an antigen or an epitope (e.g. LILBR2) alone, that is to say, without the requirement of another binding domain. In particular, the single-domain antibody according to the invention is devoid of light chain or fragment thereof. The sdAb molecules according to the present invention are polypeptides comprising or consisting of, or consisting essentially of an antigen-binding domain of a heavy chain only antibody (HcAb) which may be isolated from Camelidae, cartilaginous fish, naïve library or from an engineered form of a heavy variable domain of an antibody. Preferably, the sdAb is derived from a camelid HCAb, preferably from an alpaca HCAb.

In some preferred embodiments, the single-domain antibody is selected from the group consisting of VHH, V-NAR from Ig-NAR, engineered V-NAR, VHH variants, in particular humanized VHH or optimized VHH, and combination thereof.

In one embodiment, the sdAb against LILRB2 is an optimized sdAb. An optimized sdAb refers to a variant of a sdAb derived from an isolated HCAb which comprises one or several amino acid modifications as compared to a naturally-occurring sdAb, said modifications enabling for instance to increase the stability of the sdAb or to increase the affinity and/or the selectivity of the sdAb variant for LILRB2.

In another or further embodiment, the sdAb against LILRB2 is a humanized sdAb. A humanized sdAb refers to a sdAb variant which comprises one or several amino acid modifications as compared to a naturally-occurring sdAb, said modifications enabling to decrease its immunogenicity with respect to a human subject without significantly decreasing the affinity for LILRB2. A humanized sdAb according to the present invention may be obtained by replacing one or more of the amino acids in the Camelidae or cartilaginous fish sdAb sequence by their human counterpart, preferably as found in a human consensus sequence, with proviso that said amino acid modification does not significantly affect the antigen binding capacity of the resulting sdAb, nor its properties, such as the capacity of inhibiting the interaction between LILRB2 and human leukocyte antigen-G (HLA-G). Such a method is well-known by the skilled artisan. The state in the art provides several examples of humanized scaffold for VHHs which can be used in the context of the invention. Humanized sdAbs encompass partially humanized sdAbs and fully-humanized sdAbs.

Potentially useful humanizing amino acid modifications, in particular substitutions, can be determined by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se) and the resulting humanized VHH sequences can be tested for affinity for the target, for stability, for ease and level of expression, and/or for other desired properties. In this way, by means of a limited degree of trial and error, suitable humanizing substitutions (or suitable combinations thereof) can be determined by the skilled artisan. As an alternative, the one skilled in the art may graft the CDRs of a VHH within a humanized scaffold of VHH described in the state in the art, so as to obtain the desired humanized sdAb directed against LILRB2. Method for humanizing sdAb as well as humanized sdAb scaffolds are provided, for instance, in patent application US 2010/0215664, WO2011/117423, or in publications such as Conrath et al., Journal of Molecular Biology, 2005, 350:112-125 and in Vincke, Journal of Biological Chemistry, 2009, 284, 3273-3284.

As an alternative, the one skilled in the art may graft the CDRs within a universal scaffold of sdAb described in the state in the art (Saerens et al., J. Mol. Biol. (2005) 352, 597-607), so as to obtain the desired sdAb directed against the LILRB2. The sdAb of the invention may be a VHH comprising a universal framework scaffold, for instance as shown in Saerens et al. and comprising at least one CDR, preferably three CDRs as defined hereafter.

The single-domain antibody of the invention comprises at least one, preferably three, complementarity determining regions (CDR) which determine its binding specificity. Preferably, the single-domain antibody comprises several, preferably 3 CDRs, which are distributed between framework regions (FRs). CDRs and FRs are preferably fragments, variants or derivatives from a naturally-occurring antibody variable domain. The CDRs have generally a length of 5 to 30 amino acids and show high variability both in sequence content and structure conformation, which are involved in antigen binding and provide antigen specificity.

Preferably, the single domain antibody comprises four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively. These framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. These framework regions and complementary determining regions are preferably operably linked in the following order: FR1-CDR1-FR2-CDR2-FR3-CDR3-FR4 (from amino terminus to carboxy terminus).

The CDRs of a given sdAb can be determined by any method available to those skilled in the art. For example, and in a non-limiting manner, the Chlothia or the Kabat method can be used to determine the CDRs (Chothia et al., Nature 342, 877-883; Kabat et al., 1991, Sequences of Proteins of Immunological Interest, 5th Ed., United States Public Health Service, National Institutes of Health, Bethesda). Alternative method of determining CDRs can also be used such as the intermediate method between Chlothia and Kabat called AbM (Oxford Molecular AbM antibody modeling software) or the so-called “Contact” method based on an analysis of available complex structures (Saerens et al, Mol Biol. 2005) or on the IMGT method, such as disclosed in Lefranc et al., Dev. Comp. Immunol., 2003, 27:55-77 (“IMGT” numbering scheme).

Compared to conventional human antibody VH, a few amino acids can be substituted in the FR2 region and CDRs of sdAb. For instance, highly conserved hydrophobic amino acids (such as Val47, Gly49, Leu50, and/or Trp52) in FR2 region are often replaced by hydrophilic amino acids (Phe42, Glu49, Arg50, Gly52), rendering the overall structure more hydrophilic and contributing to high stability, solubility and resistance to aggregation.

In some particular embodiments, the single-domain antibody of the invention comprises a CDR3 which comprises, or consists in the sequence set forth in SEQ ID NO: 3, 6, 9, 12, 15, 18, 21, 24, 27, 30 or 33 or comprises, or consists in an amino acid sequence which differs from 3, 6, 9, 12, 15, 18, 21, 24, 27, 30 or 33 in virtue of one, two, or three amino acid modifications. Preferably, the single-domain antibody of the invention comprises a CDR3 which comprises, or consists in the sequence set forth in SEQ ID NO: 3, 6 or 9 or comprises, or consists in an amino acid sequence which differs from SEQ ID NO: 3, 6 or 9 in virtue of one, two, or three amino acid modifications. Preferably, such amino acid modifications do not significantly affect the antigen binding capacity of the resulting sdAb, nor its properties, such as the capacity of inhibiting the interaction between LILRB2 and human leukocyte antigen-G (HLA-G). Preferably, such amino acid modifications are substitutions such as silent substitutions.

In some particular embodiments, the single-domain antibody of the invention comprises a CDR2 which comprises, or consists in the sequence set forth in SEQ ID NO: 2, 5, 8, 11, 14, 17, 20, 23, 26, 29 or 32 or has an amino acid sequence which differs from SEQ ID NO: 2, 5, 8, 11, 14, 17, 20, 23, 26, 29 or 32 in virtue of one, two, or three amino acid modifications. Preferably, the single-domain antibody of the invention comprises a CDR2 which comprises, or consists in the sequence set forth in SEQ ID NO: 2, 5 or 8 or comprises, or consists in an amino acid sequence which differs from SEQ ID NO: 2, 5 or 8 in virtue of one, two, or three amino acid modifications. Preferably, such amino acid modifications do not significantly affect the antigen binding capacity of the resulting sdAb, nor its properties, such as the capacity of inhibiting the interaction between LILRB2 and human leukocyte antigen-G (HLA-G). Preferably, such amino acid modifications are substitutions such as silent substitutions.

In some particular embodiments, the single-domain antibody of the invention comprises a CDR1 which comprises, or consists in the sequence set forth in SEQ ID NO: 1, 4, 7, 10, 13, 16, 19, 22, 25, 28 or 31 or has an amino acid sequence which differs from 1, 4, 7, 10, 13, 16, 19, 22, 25, 28 or 31 in virtue of one, two, or three amino acid modifications. Preferably, the single-domain antibody of the invention comprises a CDR1 which comprises, or consists in the sequence set forth in SEQ ID NO: 1, 4 or 7 or comprises, or consists in an amino acid sequence which differs from SEQ ID NO: 1, 4 or 7 in virtue of one, two, or three amino acid modifications. Preferably, such amino acid modifications do not significantly affect the antigen binding capacity of the resulting sdAb, nor its properties, such as the capacity of inhibiting the interaction between LILRB2 and human leukocyte antigen-G (HLA-G). Preferably, such amino acid modifications are substitutions such as silent substitutions.

In some particular embodiments, the single-domain antibody of the invention comprises three CDRs which comprises, or consists in:

(a) CDR1 comprises, or is of, SEQ ID NO:1 or has an amino acid sequence which differs from SEQ ID NO:1 in virtue of one, two, or three amino acid modifications, and

CDR2 comprises, or is of, SEQ ID NO:2 or has an amino acid sequence which differs from SEQ ID NO:2 in virtue of one, two, or three amino acid modifications, and

CDR3 comprises, or is of, SEQ ID NO:3 or has an amino acid sequence which differs from SEQ ID NO:3 in virtue of one, two, three or four amino acid modifications; or

(b) CDR1 comprises, or is of, SEQ ID NO:4 or has an amino acid sequence which differs from SEQ ID NO:4 in virtue of one, two, or three amino acid modifications, and

CDR2 comprises, or is of, SEQ ID NO:5 or has an amino acid sequence which differs from SEQ ID NO:5 in virtue of one, two, or three amino acid modifications, and

CDR3 comprises, or is of, SEQ ID NO:6 or has an amino acid sequence which differs from SEQ ID NO:6 in virtue of one, two, three or four amino acid modifications; or

(c) CDR1 comprises, or is of, SEQ ID NO:7 or has an amino acid sequence which differs from SEQ ID NO:7 in virtue of one, two, or three amino acid modifications, and

CDR2 comprises, or is of, SEQ ID NO:8 or has an amino acid sequence which differs from SEQ ID NO:8 in virtue of one, two, or three amino acid modifications, and

CDR3 comprises, or is of, SEQ ID NO:9 or has an amino acid sequence which differs from SEQ ID NO:9 in virtue of one, two, three or four amino acid modifications; or

(d) CDR1 comprises, or is of, SEQ ID NO:10 or has an amino acid sequence which differs from SEQ ID NO:10 in virtue of one, two, or three amino acid modifications, and

CDR2 comprises, or is of, SEQ ID NO:11 or has an amino acid sequence which differs from SEQ ID NO:11 in virtue of one, two, or three amino acid modifications, and

CDR3 comprises, or is of, SEQ ID NO:12 or has an amino acid sequence which differs from SEQ ID NO:12 in virtue of one, two, three or four amino acid modifications; or

(e) CDR1 comprises, or is of, SEQ ID NO:13 or has an amino acid sequence which differs from SEQ ID NO:13 in virtue of one, two, or three amino acid modifications, and

CDR2 comprises, or is of, SEQ ID NO:14 or has an amino acid sequence which differs from SEQ ID NO:14 in virtue of one, two, or three amino acid modifications, and

CDR3 comprises, or is of, SEQ ID NO:15 or has an amino acid sequence which differs from SEQ ID NO:15 in virtue of one, two, three or four amino acid modifications; or

(f) CDR1 comprises, or is of, SEQ ID NO:16 or has an amino acid sequence which differs from SEQ ID NO:16 in virtue of one, two, or three amino acid modifications, and

CDR2 comprises, or is of, SEQ ID NO:17 or has an amino acid sequence which differs from SEQ ID NO:17 in virtue of one, two, or three amino acid modifications, and

CDR3 comprises, or is of, SEQ ID NO:18 or has an amino acid sequence which differs from SEQ ID NO:18 in virtue of one, two, three or four amino acid modifications; or

(g) CDR1 comprises, or is of, SEQ ID NO:19 or has an amino acid sequence which differs from SEQ ID NO:19 in virtue of one, two, or three amino acid modifications, and

CDR2 comprises, or is of, SEQ ID NO:20 or has an amino acid sequence which differs from SEQ ID NO:20 in virtue of one, two, or three amino acid modifications, and

CDR3 comprises, or is of, SEQ ID NO:21 or has an amino acid sequence which differs from SEQ ID NO:21 in virtue of one, two, three or four amino acid modifications; or

(h) CDR1 comprises, or is of, SEQ ID NO:22 or has an amino acid sequence which differs from SEQ ID NO:22 in virtue of one, two, or three amino acid modifications, and

CDR2 comprises, or is of, SEQ ID NO:23 or has an amino acid sequence which differs from SEQ ID NO:23 in virtue of one, two, or three amino acid modifications, and

CDR3 comprises, or is of, SEQ ID NO:24 or has an amino acid sequence which differs from SEQ ID NO:24 in virtue of one, two, three or four amino acid modifications; or

(i) CDR1 comprises, or is of, SEQ ID NO:25 or has an amino acid sequence which differs from SEQ ID NO:25 in virtue of one, two, or three amino acid modifications, and

CDR2 comprises, or is of, SEQ ID NO:26 or has an amino acid sequence which differs from SEQ ID NO:26 in virtue of one, two, or three amino acid modifications, and

CDR3 comprises, or is of, SEQ ID NO:27 or has an amino acid sequence which differs from SEQ ID NO:27 in virtue of one, two, three or four amino acid modifications; or

(j) CDR1 comprises, or is of, SEQ ID NO:28 or has an amino acid sequence which differs from SEQ ID NO:28 in virtue of one, two, or three amino acid modifications, and

CDR2 comprises, or is of, SEQ ID NO:29 or has an amino acid sequence which differs from SEQ ID NO:29 in virtue of one, two, or three amino acid modifications, and

CDR3 comprises, or is of, SEQ ID NO:30 or has an amino acid sequence which differs from SEQ ID NO:30 in virtue of one, two, three or four amino acid modifications; or

(k) CDR1 comprises, or is of, SEQ ID NO:31 or has an amino acid sequence which differs from SEQ ID NO:31 in virtue of one, two, or three amino acid modifications, and

CDR2 comprises, or is of, SEQ ID NO:32 or has an amino acid sequence which differs from SEQ ID NO: 32 in virtue of one, two, or three amino acid modifications, and

CDR3 comprises, or is of, SEQ ID NO:33 or has an amino acid sequence which differs from SEQ ID NO:33 in virtue of one, two, three or four amino acid modifications.

Preferably, the anti-LILRB2 sdAb comprises three CDRs in which:

(a) CDR1 comprises, or is of, SEQ ID NO:1 or has an amino acid sequence which differs from SEQ ID NO:1 in virtue of one, two, or three amino acid modifications, and

CDR2 comprises, or is of, SEQ ID NO:2 or has an amino acid sequence which differs from SEQ ID NO:2 in virtue of one, two, or three amino acid modifications, and

CDR3 comprises, or is of, SEQ ID NO:3 or has an amino acid sequence which differs from SEQ ID NO:3 in virtue of one, two, three or four amino acid modifications; or

(b) CDR1 comprises, or is of, SEQ ID NO:4 or has an amino acid sequence which differs from SEQ ID NO:4 in virtue of one, two, or three amino acid modifications, and

CDR2 comprises, or is of, SEQ ID NO:5 or has an amino acid sequence which differs from SEQ ID NO:5 in virtue of one, two, or three amino acid modifications, and

CDR3 comprises, or is of, SEQ ID NO:6 or has an amino acid sequence which differs from SEQ ID NO:6 in virtue of one, two, three or four amino acid modifications; or

(c) CDR1 comprises, or is of, SEQ ID NO:7 or has an amino acid sequence which differs from SEQ ID NO:7 in virtue of one, two, or three amino acid modifications, and

CDR2 comprises, or is of, SEQ ID NO:8 or has an amino acid sequence which differs from SEQ ID NO:8 in virtue of one, two, or three amino acid modifications, and

CDR3 comprises, or is of, SEQ ID NO:9 or has an amino acid sequence which differs from SEQ ID NO:9 in virtue of one, two, three or four amino acid modification.

Preferably, such amino acid modifications do not significantly affect the antigen binding capacity of the resulting sdAb, nor its properties, such as the capacity of inhibiting the interaction between LILRB2 and human leukocyte antigen-G (HLA-G). Preferably, such amino acid modifications are substitutions such as silent substitutions.

Even more preferably, the anti-LILRB2 sdAb comprises three CDRs in which CDR1 comprises, or is of, SEQ ID NO:1 or has an amino acid sequence which differs from SEQ ID NO:1 in virtue of one, two, or three amino acid modifications, preferably one, two, or three silent mutations, even more preferably one, two, or three silent substitutions, and CDR2 comprises, or is of, SEQ ID NO:2 or has an amino acid sequence which differs from SEQ ID NO:2 in virtue of one, two, or three amino acid modifications, preferably one, two, or three silent mutations, even more preferably one, two, or three silent substitutions, and CDR3 comprises, or is of, SEQ ID NO:3 or has an amino acid sequence which differs from SEQ ID NO:3 in virtue of one, two, three or four amino acid modifications, preferably one, two, or three silent mutations, even more preferably one, two, or three silent substitutions.

In some embodiments, the anti-LILRB2 sdAb comprises or consists essentially of a sequence defined in any of the sequence SEQ ID No: 34 to SEQ ID No: 44 or a sequence having at least 80% sequence identity thereto, preferably at least 90%, 92%, 94%, 95%, 96%, 97%, 98%, 99% or more amino-acid sequence identity thereto.

Preferably, the anti-LILRB2 sdAb comprises or consists in a sequence selected in the group consisting of SEQ ID NO: 34, SEQ ID NO: 35 and SEQ ID NO: 36 or a sequence having at least 80% sequence identity thereto, preferably at least 90%, 92%, 94%, 95%, 96%, 97%, 98%, 99% or more amino-acid sequence identity thereto.

In one embodiment, the anti-LILRB2 sdAb comprises or consists in a sequence defined in SEQ ID NO: 34 or a sequence having at least 80% sequence identity thereto, preferably at least 90%, 92%, 94%, 95%, 96%, 97%, 98%, 99% or more amino-acid sequence identity thereto. Preferably, the anti-LILRB2 sdAb that comprises or consists in a sequence having at least 80%, 90%, 92%, 94%, 95%, 96%, 97%, 98%, 99% or more amino-acid sequence identity to the SEQ ID NO:34 is still able to bind to LILRB2, preferably with a affinity similar to the anti-LILRB2 sdAb that comprises or consists in a sequence defined in SEQ ID NO: 34, and conserves the same properties, such as the capacity of inhibiting the interaction between LILRB2 and human leukocyte antigen-G (HLA-G).

In some particular embodiments, the sdAb of the invention has a molecular weight from about 11 kDa to about 18 kDa, for instance from 11 kDa to 17 kDa such as from 14 to 16 kDa or from 14.5 to 15.5 kDa such as about 15 kDa.

In certain aspects, the sdAb binds LILRB2 with affinities of at least about 10⁻⁶ M or 10⁻⁷ M, and preferably at least, 10⁻⁸ M, 10⁻⁹ M 10⁻¹⁰ M or 10⁻¹¹ M. Particularly, the apparent K_{d} is comprised between 0.1 nM and 10 μM, particularly between 1 μM and 1 nM. The binding affinity can be measured by any method available to the person skilled in the art, in particular by surface plasmon resonance (SPR).

In a preferred embodiment, the anti-LILRB2 sdAb does not recognize other member of the LILBR family other than LILRB2. Preferably, the anti-LILRB2 sdAb does not recognize LILRB1. Alternatively, the anti-LILRB2 sdAb recognize weakly LILRB1. Preferably, the anti-LILRB2 sdAb recognize less LILRB1 than LILRB2, particularly by a factor 10, 100 or 1000.

In a particular embodiment, the anti-LILRB2 sdAb competitively inhibits the interaction between LILRB2 and human leukocyte antigen-G (HLA-G) or competitively inhibits the binding of human leukocyte antigen-G (HLA-G) to LILRB2.

The term “competitively inhibits” indicates that the sdAb according to the invention can reduce or inhibit or displace the binding of a protein, antibody or ligand to LILRB2, or the interaction between any protein, antibody or ligand and LILRB2, particularly in vitro, ex vivo or in vivo. Competition assays can be performed using standard techniques such as, for instance, competitive ELISA or other binding assays. When a sdAb inhibits or displaces at least 30%, 40%, 50%, 60%, 70% or 80% of the binding of the protein, antibody or ligand to LILRB2, it is considered as competitive. Preferred competing sdAbs bind epitopes that share common amino acid residues with the epitopes recognized or bound by the protein, antibody or ligand on LILRB2.

As used herein, the term “HLA-G” designates the Human leukocyte antigen G which includes at least seven isoforms, where four are membrane-bound (HLA-G1, HLA-G2, HLA-G3 and HLA-G4) and three are soluble (HLA-G5, HLA-G6 and HLA-G7). HLA-G human isoforms are for example described under the Uniprot accession number P17693-1 for HLA-G1, P17693-2 for HLA-G2, P17693-3 for HLA-G3, P17693-4 for HLA-G4, P17693-5 for HLA-G5, P17693-6 for HLA-G6, P17693-7 for HLA-G7.

In a particular embodiment, the anti-LILRB2 sdAb competitively inhibits the interaction between LILRB2 and HLA-G6 or competitively inhibits the binding of HLA-G6 to LILRB2.

In another embodiment, the sdAb according to the invention competitively inhibits the binding of Angiopoietin Like 2 (ANGPTL2) to LILRB2 or competitively inhibits the interaction between LILRB2 and ANGPTL2. As used herein, “ANGPTL2” is a member of the vascular endothelial growth factor family which is known in the art for its pro-angiogenic and antiapoptotic capacities. This term preferably refers to human ANGPTL2. Human ANGPTL2 is for example described under the Uniprot accession number O15123.

The invention also relates to chimeric agents (also interchangeably called herein “conjugates”) comprising one or more anti-LILRB2 sdAb as defined above, conjugated to at least one molecule. The molecule conjugated to sdAb may be for example any active compound useful in medicine, such as a drug, an imaging molecule, a diagnostic agent, a tracer, a tag or a dye. The chimeric agent may also contain, in addition to or instead of said active compound, a stabilizing group (e.g., a Fc or IgG for instance) to increase the plasma half-life of the sdAb or conjugate. Such chimeric agent can be prepared using a coupling between a sdAb and a molecule by any methods known in the art, preferably by a chemical, biochemical or enzymatic pathway, or by genetic engineering.

In a particular embodiment, the anti-LILRB2 sdAb of the invention may be fused or conjugated to a labelling mean, e.g. a molecule or a protein selected from an enzyme such as horseradish peroxidase or alkaline phosphatase, a fluorescent protein such as GFP, a fluorescent label such as fluorescein rhodamine, label, a chemiluminescent label or bioluminescent label such as luminal, a chromophore, a radio-isotope e.g. suitable for in vivo, ex vivo or in vitro imaging or diagnosing.

In another particular embodiment, the sdAb according to the invention is comprised into a CAR construct. The terms “Chimeric antigen receptor” (CAR), “engineered cell receptor”, or “chimeric immune receptor” (ICR) as used herein refer to engineered receptors, which graft an antigen binding specificity onto immune cells, thus combining the antigen binding properties of the antigen binding domain with the immunogenic activity of the immune cell, such as the lytic capacity and self-renewal of T cells. Particularly, a CAR refers to a fused protein comprising optionally a signal peptide, an extracellular domain able to bind an antigen, a transmembrane domain, optionally a hinge domain and at least one intracellular domain. In a preferred embodiment, the CAR comprises an anti-LILRB2 sdAb as disclosed herein as the extracellular or antigen binding domain, a transmembrane domain, optionally a hinge domain and at least one intracellular domain.

Nucleic Acids, Vectors and Host Cells

A further aspect of the invention relates to an isolated nucleic acid construct or a polypeptide construct encoding a sdAb as defined above. The nucleic acid may be single- or double-stranded or a mixture of the two. The nucleic acid can be DNA (cDNA or gDNA), RNA, or a mixture thereof. It can comprise modified nucleotides, comprising for example a modified bond, a modified purine or pyrimidine base, or a modified sugar. It can be prepared by any method known to one skilled in the art, including chemical synthesis, recombination, and/or mutagenesis.

The nucleic acid according to the invention may be deduced from the amino acid sequence of the sdAb molecules according to the invention and codon usage may be adapted according to the host cell in which the nucleic acid shall be transcribed. These steps may be carried out according to methods well known to one of skill in the art and some of which are described in the reference manual Sambrook et al. (Sambrook J, Russell D (2001) Molecular cloning: a laboratory manual, Third Edition Cold Spring Harbor). Specific examples of such nucleic acid sequences include the sequences comprising anyone of SEQ ID NOs: 61-75, and the complementary sequence thereto.

The invention also relates to a vector containing such an isolated nucleic acid, optionally under control of regulatory sequences (e.g., promoter, terminator, etc.). The vector may be for example a plasmid, virus, cosmid, phagemid or artificial chromosome.

The present invention further relates to the use of a nucleic acid or vector according to the invention to transform, transfect or transduce a host cell.

The present invention thus also provides a host cell comprising one or several nucleic acids of the invention and/or one or several vectors of the invention and/or one or several polypeptides encoding the sdAb of the invention.

The host cell may be any host cell capable of expressing or producing a sdAb of the invention, including e.g. a prokaryotic host cell, such as e.g., E. coli, or a (cultured) mammalian, plant, insect, fungal or yeast host cell, including e.g. CHO-cells, BHK-cells, human cell lines (including HeLa, COS and PER C6), Sf9 cells and Sf+ cells. An appropriate host cell encompasses a cell of a eukaryotic microorganism such as yeasts and filamentous fungi. Preferred yeast host cell includes Saccharomyces cerevisiae, Pichia pastoris, Hansenula polymorpha, and Kluyveromyces lactis. The term “host cell” also encompasses any progeny of a parent host cell that is not identical to the parent host cell due to mutations that occur during replication. Preferably, the cell is not a human embryonic stem cell.

A further object of the invention is a method for producing a sdAb according to the invention, wherein the method comprises the steps of:

a) culturing a host cell as previously-defined and

b) recovering the said nucleic acid, vector or polypeptide encoding the sdAb as defined hereabove from the cell culture.

It goes without saying that step a) is performed under conditions allowing the expression of the desired nucleic acid, vector or polypeptide by the host cell. Suitable expression conditions may include the use of a suitable medium, the presence of a suitable source of food and/or suitable nutrients, a suitable temperature, and optionally the presence of a suitable inducing factor or compound (e.g. when the nucleotide sequences of the invention are under the control of an inducible promoter); all of which may be selected by the skilled artisan in the art.

Under such conditions, the sdAb of the invention may be expressed in a constitutive manner, in a transient manner, or only when suitably induced.

The sdAb of the invention may then be isolated from the host cell and/or from the culture medium in which said host cell was cultivated, using protein isolation and/or purification techniques known per se, such as chromatography and/or electrophoresis techniques, differential precipitation techniques, affinity techniques and the like. The sdAb may also comprise a tag such as a histidine or a streptavidin tag for purification purposes.

The invention also provides a method to obtain a sdAb against LILRB2 as defined herein. The method for obtaining and/or selecting a sdAb according to the invention may be based on a protein selection technology such as, but without being limited to, cell display, phage display, ribosome display, mRNA display, DNA display or plasmid display. These techniques are well-described in the state in the art. For instance, in order to generate a library of VHHs displayed on bacteriophages, the skilled artisan can refer to Muydermans et al., Molecular Biotechnology, 2001, 74, 277-302, in particular to the section entitled Recombinant VHH, the disclosure of which being incorporated therein by reference. In order to generate a library of V-NARs displayed on bacteriophages, the skilled artisan may refer to Dooley et al. Mol Immunol, 2003, 40:25-30. In certain embodiments, the method of the invention may encompass one or several steps enable to select functional sdAbs, in particular sdAbs which are able to recognize LILRB2 or which competitively inhibit the interaction between LILRB2 and HLA-G, and/or which competitively inhibit the interaction between LILRB2 and ANGPTL2.

In a particular embodiment, the CAR comprising a sdAb according to the invention is expressed by a cell. The cell can be a prokaryotic or a eukaryotic cell. Preferably, the cells are eukaryotic cells, such as mammalian cells. Preferably, the cells expressing the CAR comprising the sdAb according to the invention are immune cells. The cells can be selected from a group consisting of a macrophage, a T cell, a B cell, a NK cell, a NKT, monocyte and dendritic cell. Preferably, the cell is not a human embryonic stem cell.

Pharmaceutical Composition

The invention also relates to a pharmaceutical composition characterized in that it comprises at least one sdAb, CAR or cell as defined above and optionally one or more pharmaceutically acceptable excipients.

The pharmaceutical composition of the invention may be formulated according to standard methods such as those described in Remington: The Science and Practice of Pharmacy (Lippincott Williams & Wilkins; Twenty first Edition, 2005). Pharmaceutically acceptable excipients that may be used are, in particular, described in the Handbook of Pharmaceuticals Excipients, American Pharmaceutical Association (Pharmaceutical Press; 6th revised edition, 2009).

In one aspect, the compositions of the invention advantageously comprise a pharmaceutically acceptable carrier or excipient. The pharmaceutically acceptable carrier can be selected from the carriers classically used according to each mode of administration such as (a) fillers or diluents such as for example, starch, lactose, sucrose, glucose, mannitol, microcrystalline cellulose and silicic acid; (b) binders, such as, carboxymethylcellulose, gelatin, polyvinylpyrrolidone, sucrose; (c) humectants, as for example, glycerol; (d) disintegrating agents, as for example, agar-agar, calcium carbonate, potato or tapioca starch, alginic acid, certain complex silicates, sodium croscarmellose and sodium carbonate; (e) solution retarders, as for example paraffin; (f) absorption accelerators, such as quaternary ammonium compounds; (g) wetting agents, such as glycerol monostearate; (h) adsorbents such as kaolin and bentonite; (i) lubricants such as talc, calcium stearate, magnesium stearate, solid polyethylene glycols, sodium lauryl sulfate, (j) antioxidant agents, (k) buffering agents such as sodium citrate or sodium phosphate, (l) preservatives, (m) flavors and perfumes, etc.

The pharmaceutical composition of the invention may be obtained by admixing a sdAb, a CAR, a cell or a polypeptide of the invention with an appropriate degree of purity with at least one customary excipient (or carrier) as described hereabove. In particular, a sdAb, a CAR, a cell or a polypeptide of the invention is the active ingredient of the composition.

It goes without saying that the excipient(s) to be combined with the active ingredient may vary upon (i) the physico-chemical properties including the stability of the said active ingredient, (ii) the pharmacokinetic profile desired for said active ingredient, (iii) the galenic form and (iv) the route of administration.

The pharmaceutical compositions typically comprise an effective dose of a sdAb, a CAR, or a cell of the invention. A “therapeutically effective dose” as described herein refers to the dose that gives a therapeutic effect for a given condition and administration schedule. A “therapeutically effective dose” of an active substance does not necessarily cure a disease or disorder but will provide a treatment for this disease or disorder so that its appearance is delayed, impeded or prevented, or its symptoms are attenuated, or its term is modified or is less severe, or the recovery of the patient is accelerated.

The pharmaceutical compositions of the invention may be formulated to be suitable for administration by any conventional route, including by enteral route (i.e. oral) e.g. in the form of tablets, capsules, by parenteral, intramuscular, transdermal, intravenous route e.g. in the form of injectable solutions or suspensions and by topical route e.g. in the form of gels, ointments, gels, lotions, patches, suppositories and the like.

In some particular embodiments, the pharmaceutical composition may be a lyophilizate or a freeze-dried powder which may be dissolved in an appropriate vehicle just before being administered to the subject.

The invention also relates to a diagnostic composition characterized in that it comprises a sdAb or sdAb-diagnostic or medical imaging agent conjugate compound such as defined above.

### Uses According to the Invention

The sdAbs, the CARs, the cells, the compositions and the constructs (i.e. isolated nucleic acids, polypeptides and/or vectors) according to the invention may be used in various fields, including biological research, biochemical industry or medicine.

Particularly the sdAbs, the CARs, the cells, the compositions and the constructs of the present invention find application in subjects having or suspected of having a cancer, particularly for reducing the size of a tumor or preventing the growth or re-growth of a tumor in these subjects or preventing the induction of an immunosuppressive microenvironment.

In one embodiment, the subject to treat is a non-human animal, in particular a mammal such as dogs, cats, horses, cows, pigs, sheep and non-human primates. Alternatively, the subject to treat may be a human, particularly a human, at any age, including a child, an adolescent or an adult.

Particularly, the subject is affected with a disease that involve LILBR2 expression, particularly LILBR2 over-expression. In one embodiment, the subject is suffering from cancer, an inflammatory disorder, an infectious disease for example such as caused by a bacterium, a virus or a fungus, or from an auto-immune disease.

Preferably, the subject is suffering from cancer, even more preferably from a LILBR2 positive cancer. For example, a subject suitable for the treatment of a disease such as cancer, can be identified by examining whether such a subject carries LILRB2 positive cells, particularly LILRB2 positive cancer cells, preferably such cells overexpressing LILRB2. Examples of diseases and cancers are more particularly described hereafter.

A further object of the invention is a sdAb, a CAR, a cell, a polypeptide construct or a pharmaceutical composition according to the invention for use in the treatment of a disorder or disease involving a LILRB2 receptor, preferably such as cancer, and/or for use as a medicament or vaccine. Accordingly, it is herein described methods for inhibiting the growth of a tumor or the spread of metastasis in a subject in need thereof and/or for treating a cancer in a patient in need thereof. The tumor may be a solid tumor or a liquid tumor, preferably a solid tumor. In some embodiments, the tumor or cancer expresses or overexpresses LILBR2.

In certain embodiments, these methods comprise, or alternatively consist essentially of, or yet further consist of, administering to the subject or patient a therapeutically effective amount of the sdAbs, the CAR, the cells, the compositions and the constructs of the present invention. In a further aspect, the subject has been previously selected for the therapy by a diagnostic, preferably to evaluate if the tumor expresses or overexpresses LILBR2.

Since human LILRB2 is a relevant target for the treatment of disease or disorder, particularly such as cancer, the anti-LILRB2 sdAbs may be used as a drug, medicament or vaccine. The sdAb, CAR, cell or polypeptide construct according to the invention can be used as a medicament or vaccine or for the manufacture of a medicament or vaccine in the treatment of a disease, disorder, or condition in a subject. In some embodiments, such a medicament or vaccine can be used for treating cancer.

In one embodiment, the sdAbs, the CAR, the cells, the compositions and the constructs of the present invention are for use in the treatment of a pathology, disease and/or disorder that could be prevented or treated by the inhibition of the binding of HLAG and/or ANGPTL2 to LILRB2. Accordingly, the invention relates to a method of treatment of a pathology, disease and/or disorder that could be prevented or treated by the inhibition of the binding of HLAG and/or ANGPTL2 to LILRB2.

The invention also relates to a method for treating a subject suffering from a disorder or disease involving a LILRB2 receptor, wherein said method comprises administering to said subject a therapeutically effective amount of a sdAb, a CAR, a cell, a construct or a pharmaceutical composition according to the invention.

In a particular embodiment, the disease or disorder is cancer, preferably solids tumors, even more preferably selected from the group consisting of lung cancer, non-small cell lung cancer (NSCLC), pancreatic cancer, pancreatic ductal carcinoma, Chronic Lymphocytic Leukemia (CLL), Acute Myeloid Leukemia (AML), endometrial cancer, hepatocellular carcinoma, melanoma, ovarian cancer, breast cancer, colorectal cancer, glioma, stomach cancer, renal cancer, testis cancer, Esophageal cancer, Cervical cancer, Lewis Lung cancer of mice, Leukemia, Thyroid cancer, Liver cancer, Urothelial cancer and Head and neck cancer.

Accordingly, the present invention also relates to methods for inhibiting the growth of a tumor in a subject in need thereof and/or for inhibiting the growth and/or spread of metastasis. The tumor may be a solid tumor, or a liquid tumor. In some embodiments, the tumor or cancer expresses or overexpresses LILRB2.

The sdAb, CAR, cell or a pharmaceutical composition described herein may be administered with other therapeutics concomitantly or subsequently, including for example, small molecules, radiation therapy, chemotherapy, surgery, particularly anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions can be provided in a combined amount effective to kill or inhibit proliferation of the cell.

Conventional methods, known to those of ordinary skill in the art of medicine, can be used to administer the sdAb, composition, construct or CAR disclosed herein to a subject, depending upon the type of diseases to be treated or the site of the disease. This composition can be administered via conventional routes, e.g., administered parenterally (e.g. by intravenous, subcutaneous, intradermal, or intramuscular route), or by oral, nasal or pulmonary route.

Use in Diagnostic and Prognostic

The single-domain antibodies may be used as ligands for the purification of LILRB2. They can also be used as crystallization chaperone so as to promote the crystallization of a LILRB2 receptor.

The sdAbs and the polypeptides of the invention may also be used in cell immuno-staining, in in vivo or in vitro imaging and for diagnosis purposes. The invention also relates to a sdAb, conjugate, or compositions as described above for use for diagnosing, imaging or treating cells expressing LILRB2, preferably over-expressing LILRB2, such as cancer cells.

They may also be used as biological reagents in in vitro assays, e.g. as test compounds or competitive binders for the identification, the screening or the characterization of potential drugs targeting a LILRB2 receptor.

The anti-LILRB2 sdAbs disclosed herein can be used diagnostically to monitor expression LILRB2 levels in tissue or cells as part of a clinical testing procedure in vitro or ex vivo as well as in vivo, e.g., to determine the efficacy of a given treatment regimen.

The detection method of the present disclosure can be used to detect levels of expression LILRB2 in a biological sample in vitro or ex vivo as well as in vivo, for example after a biopsy of an organ or tissue, to test if the cells are cancerous. In vitro or ex vivo techniques for detection of LILRB2 by the sdAbs of the invention include enzyme linked immunosorbent assays (ELISAs), RIA, EIA and other “sandwich assays”, Western blots, flow cytometry, immunoprecipitations, radioimmunoassay, and immunofluorescence (e.g., IHC). Furthermore, in vivo techniques for detection of LILRB2 polypeptides include introducing into a subject a labeled anti-LILRB2 sdAbs. In in vivo techniques for detection of LILRB2 by the sdAbs of the invention, the sdAb can be labeled with a radioactive marker whose presence and location in a subject can be detected by standard imaging techniques.

The present invention also provides diagnostic, prognostic or predictive assays for determining whether a subject is at risk of developing a medical disease or condition associated with increased LILRB2 expression or activity (e.g., detection of a precancerous or cancerous cell that overexpress LILRB2). Such assays can be used for prognostic or predictive purpose to thereby prophylactically treat an individual prior to the onset of a medical disease or condition characterized by or associated with LILBR2 expression or overexpression.

The invention also provides diagnostic prognostic or predictive assays methods, wherein the sdAb according to the invention is used to select subjects eligible for therapy with an anti-LILRB2 sdAb, e.g. where LILRB2 is a biomarker for selection of patients, for where LILRB2 is overexpressed in cells, such as tumoral cells.

Kits

Any of the sdAb, composition, CAR, cell, vector, polypeptide or nucleic acid construct described herein may be included in a kit provided by the present invention.

In certain embodiments the kit includes suitable container means, cells, buffers, cell media, vectors, primers, restriction enzymes, salts, and so forth, for example. The kits may also comprise means for containing a sterile, pharmaceutically acceptable buffer and/or other diluents.

In some embodiments, means of taking a sample from an individual and/or of assaying the sample may be provided in the kit.

In some embodiments, the kit further includes an additional agent for treating cancer or an infectious disease, and the additional agent may be combined with the sdAb, composition, CAR, cell, vector, polypeptide or nucleic acid construct, or other components of the kit of the present invention or may be provided separately in the kit.

In some cases of the invention, the kit also includes a second cancer therapy, such as chemotherapy and/or other immunotherapy, for example. The kit(s) may be tailored to a particular cancer, such as a cancer expressing or overexpressing LILRB2.

The containers may be unit doses, bulk packages (e.g., multi-dose packages) or sub-unit doses. In an embodiment, the invention relates to a kit as defined above for a single-dose administration unit. The kit of the invention may also contain a first recipient comprising a dried/lyophilized bifunctional molecule and a second recipient comprising an aqueous formulation. In certain embodiments of this invention, kits containing single-chambered and multi-chambered pre-filled syringes (e.g., liquid syringes and lyosyringes) are provided. The kits of this invention are in suitable packaging. Suitable packaging includes, but is not limited to, vials, bottles, jars, flexible packaging (e.g., sealed Mylar or plastic bags), and the like.

The instructions related to the use of the sdAb, composition, CAR, cell, vector, polypeptide or nucleic acid construct described herein generally include information as to dosage, dosing schedule, route of administration for the intended treatment, or means for reconstituting or diluting such components. Instructions supplied in the kits of the invention are typically written instructions on a label or package insert (e.g., a paper sheet included in the kit in the form of a leaflet or instruction manual). In some embodiments, the kit can comprise instructions for use in accordance with any of the methods described herein. The included instructions can comprise a description of administration of sdAb, composition, CAR, cell, vector, polypeptide or nucleic acid construct described herein, particularly in the context of the treatment of a disease as described herein such as cancer. The kit may further comprise a description of selecting an individual suitable for a treatment based on identifying whether that individual has a disease associated with the LILBR2, e.g., those described herein.

Other aspects and advantages of the present invention will become apparent upon consideration of the examples below, which are only illustrative in nature and which do not limit the scope of the present application.

### Examples

Identification of LILRB2-Fc-Specific VHHs.

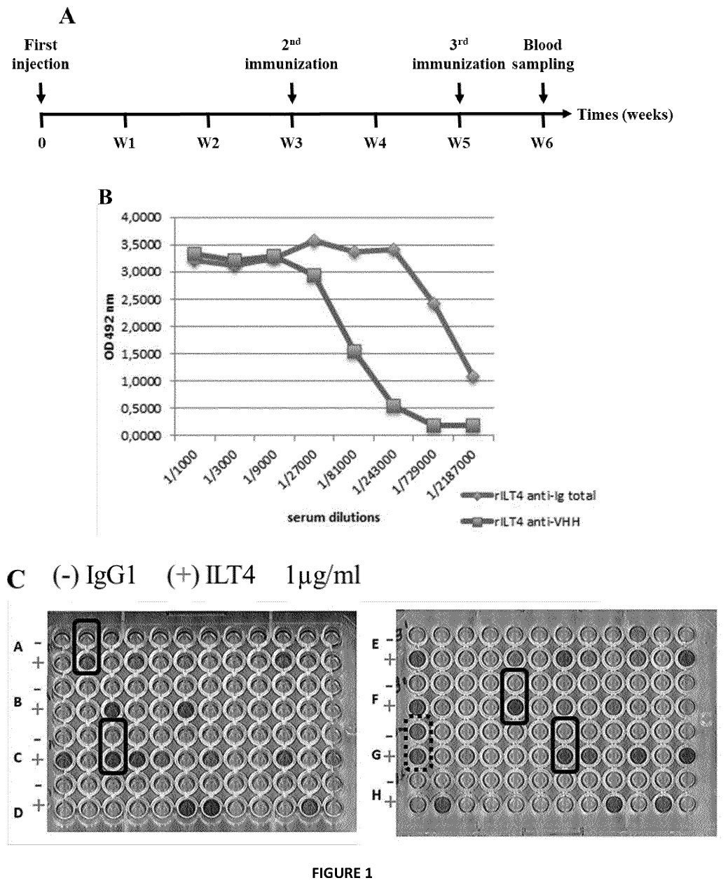

An alpaca was first immunized with LILRB2-Fc proteins in complete Freund adjuvant and subsequently boosted twice with LILRB2-Fc proteins in incomplete Freund adjuvant. The conventional antibody subclasses (i.e the IgG1), and the VHHs were fractionated from the alpaca serum. Serum was serially diluted and tested against LILRB2-Fc proteins by ELISA. Then, B lymphocytes were purified from alpaca and a library containing 3,5.10⁷ clones was obtained. Biospanning using a display against LILRB2-Fc was performed and pre-plasmic (PE)—ELISA on the selected VHHs was carried out. 400 colonies were tested and 130 positive clones against LILRB2 were identified, which are circled in full line while negative clones are circled in dotted line (FIG. 1). All positive clones were sequenced and 12 unique sequences of the VHHs were generated, and purified.

Nbs B8, C7 and C9 Recognize Linear Epitopes of rhLILRB2.

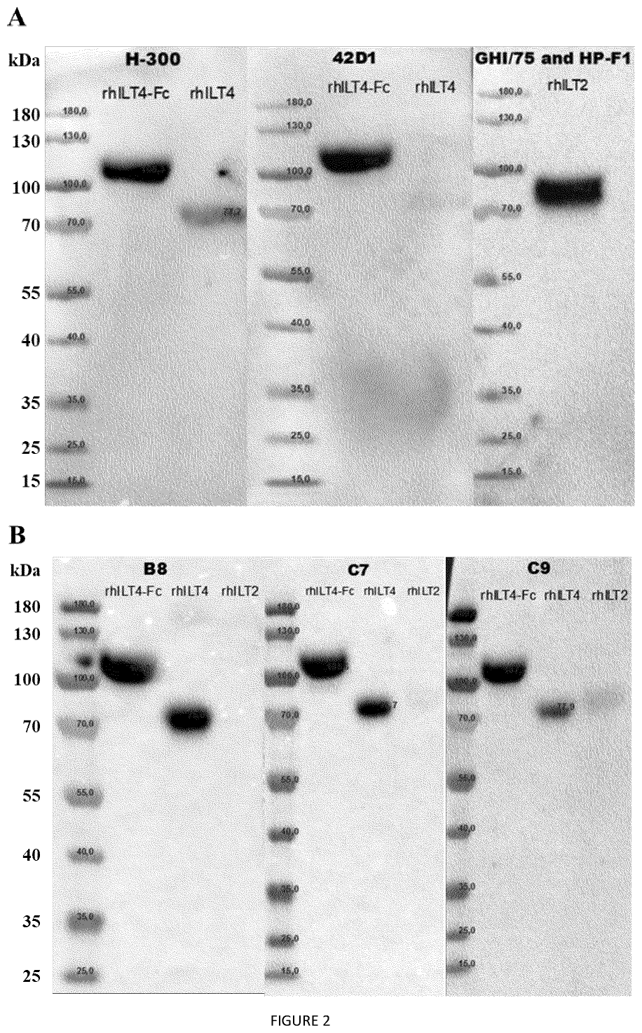

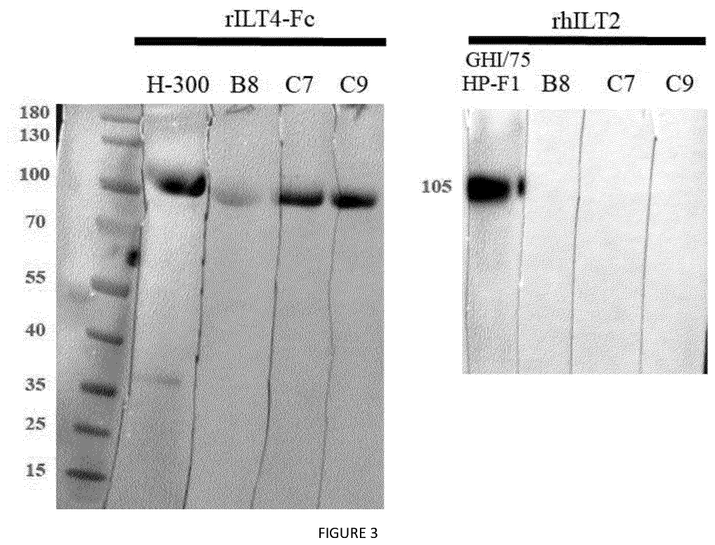

The inventors first investigated if the generated Nbs were specific for LILRB2 receptor without any cross-reactivity against LILRB1 receptor. For that, Western blotting was performed in reducing conditions. Purified dimeric rhLILRB2-Fc, monomeric rhLILRB2 and monomeric rhLILRB1 proteins were used to compare the antigen specificity of the different Nbs. Nbs specificities were evaluated against control antibodies H-300 (specific for LILRB1, -2, -4, -5, -6), 42D1 (specific for LILRB2), GHI/75 (specific for LILRB1) and HP-F1 (specific for LILRB1). The membrane labeled with H-300 polyclonal antibody (specific for LILRB1, -2, -4, -5 and -6 proteins) showed a band around 105 kDa and at 77 kDa corresponding to the size of rhLILRB2-Fc and of rhLILRB2 respectively (FIG. 2). The membrane incubated with 42D1 monoclonal antibody (specific for LILRB2-Fc receptor) displayed a unique band at 105 kDa corresponding to the size of rhLILRB2-Fc and no band at 77 kDa, demonstrating that 42D1 mAb did not recognize rhLILRB2. The membrane incubated with GHI/75 and HP-F1 monoclonal antibodies showed a band at 84 kDa corresponding to the size of rhLILRB1. Among the 15 Nbs, only B8, C7 and C9 displayed a band around 105 kDa corresponding to the molecular weight of rhLILRB2-Fc (FIG. 3) and also a band around 77 kDa corresponding to the molecular weight of D1 and D2 domains of rhLILRB2. Furthermore, incubation with B8 and C7 Nbs did not reveal any band around 84 kDa. This implies that B8 and C7 Nbs do not bind to rhLILRB1. Yet, C9 Nb displayed a weak band around 84 kDa, suggesting that C9 specificity is not completely restricted to rhLILRB2 receptor. Taken together, these data demonstrated that the Nbs were capable of recognizing the denaturated dimeric LILRB2-Fc (D1-D2-Fc) protein, which was the immunogen used to induce the Nbs, as well as the denaturated monomeric LILRB2 (D1-D2-D3-D4 domains) protein. For Western Blot experiment, c-Myc«.9E10 »pure (E-Bioscience, Ref 14-6784-82) antibody was used.

Nbs Specificity for LILRB2 Receptors on LILRB2 Transduced D1.1 Cell Line.

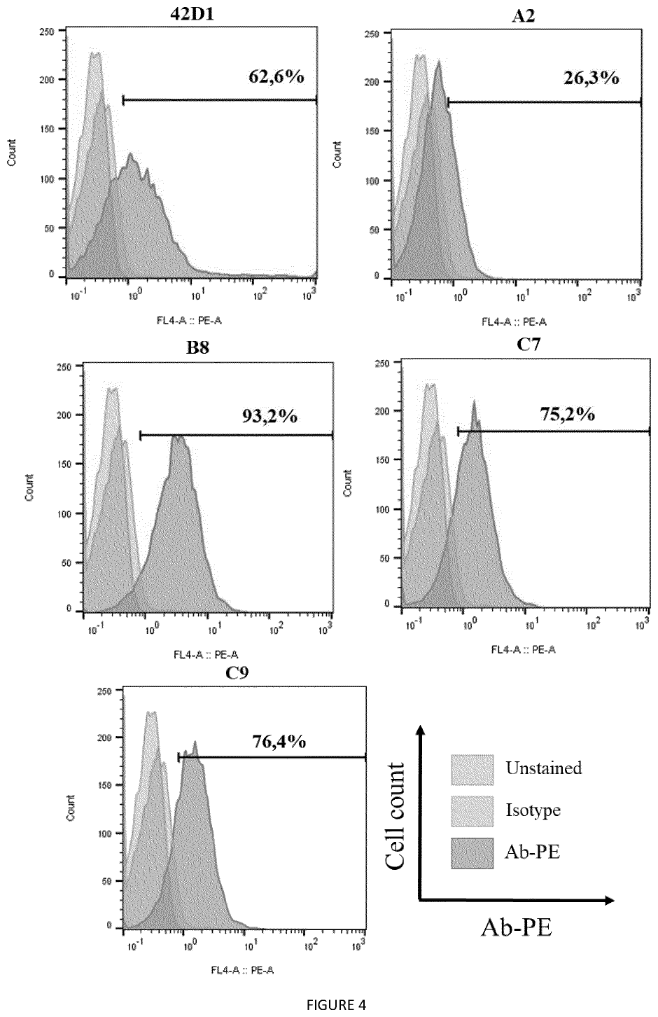

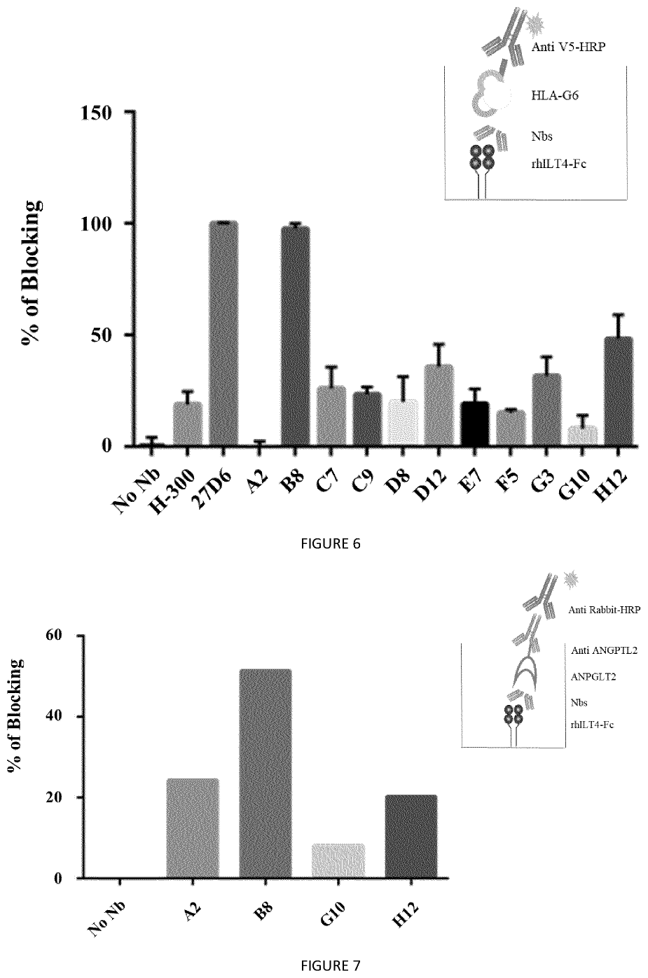

The inventors then sought to determine whether the Nbs obtained were capable of binding to conformational LILRB2 receptors. They evaluated the binding specificity of the 15 Nbs against the conformational LILRB2 receptors. For that, the inventors assessed Nbs specificity on the LILRB2-D1.1 transduced cell line generated by Invectys. For this purpose, LILRB2-D1.1 cell line was incubated with the Nbs and compared to the 42D1 control Ab. As shown on FIG. 4, 62.6% of LILRB2-D1.1 cells were labeled by 42D1 control Ab. Interestingly, 93.2%, 76.4% and 75.2% of LILRB2-D1.1 cell line was labeled by B8, C9 and C7 Nbs respectively (FIG. 7) whereas less than 40% of LILRB2-D1.1 cell line were labeled by other Nbs such as A2 (data not shown). The inventors hypothesized that the epitope recognized by B8, C7 and C9 is more accessible than the epitopes of 42D1 control antibody and of others Nbs. For flow cytometry, Mouse monoclonal antibody [9E10] to Myc tag—Phycoerythrin (Abcam, Ref: ab72468) was used.

Nbs Specificity for LILRB2 Receptors Expressed by Monocytes

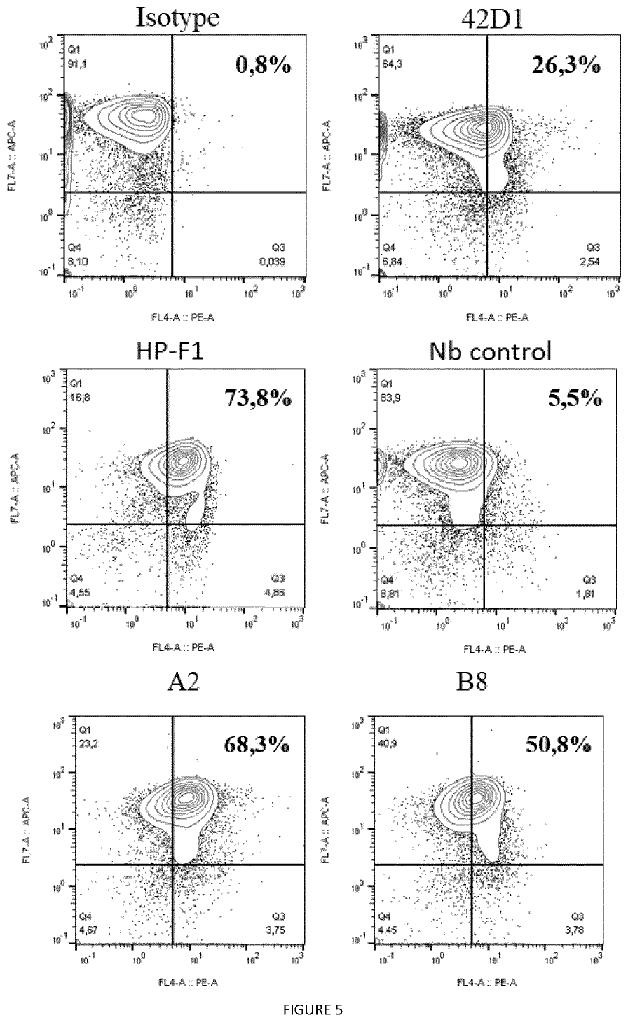

Monocytes strongly express either monomeric or dimeric LILRB2 receptors at their surface and are a relevant model to study macrophages LILRB2 expression. Thus, the inventors assessed the specificity of the anti-LILRB2 Nbs against monocytes purified from healthy donors PBMCs. Monocytes were phenotyped by labelling with anti-CD14, anti-LILRB1 Abs and the anti-LILRB2 Nbs. 38% of monocytes were positive for 42D1 control Ab and 5% were positive for an irrelevant Nb (e.g. Nb that was raised in alpaca against an antigen that is not ILT4). With anti-LILRB2 Nbs, more than 50% of monocytes were labeled: 68.3% for A2 Nb, 50, 8% for B8 Nb, 62% for C7 Nb, 58.1% for C9 Nb, 53.5% for D8 Nb, 57% for G3 Nb and 46.7% for G10 Nb (FIG. 5). Yet, monocytes were negative for D12, F5 and H12 Nbs (data not shown). Altogether, the inventors determined that B8, C7 and C9 Nbs are strongly specific for a linear epitope within LILRB2 receptors either monomeric or dimeric. The accessibility of the LILRB2 epitope for the in vitro LILRB2-D1.1 generated cell line or for ex vivo monocytes might be more difficult for control Ab 42D1 than for Nbs. This weak binding for the 42D1 monoclonal Ab is likely related to steric hindrance which does not affect anti-LILRB2 Nbs, especially B8, C7 and C9 Nbs.

Nb Anti-LILRB2 Inhibits LILRB2/HLA-G Interaction.

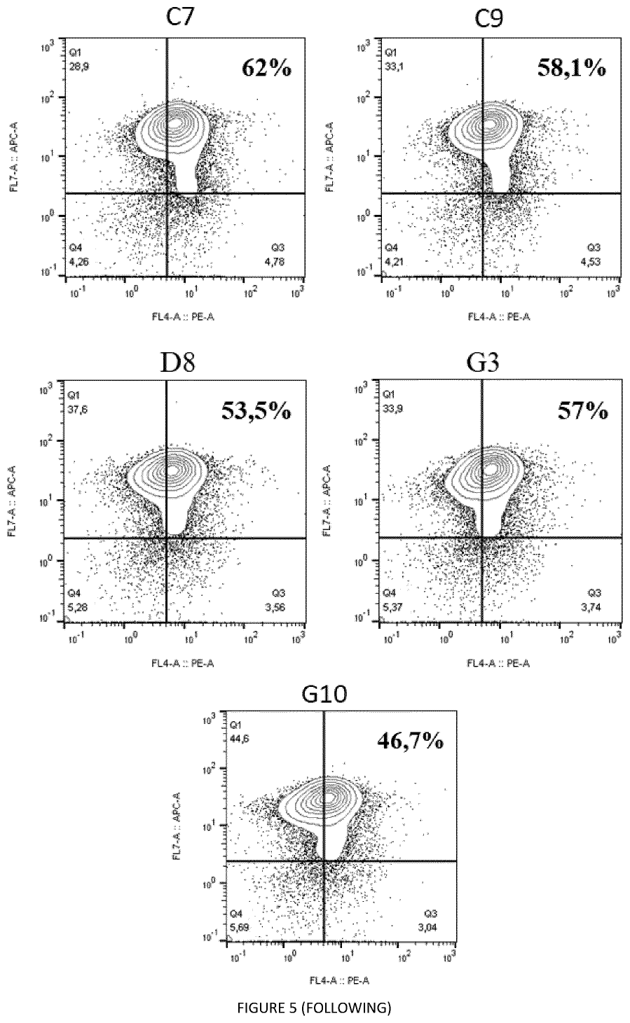

LILRB2 receptors interact either with HLA-G and ANGPTL2 to inhibit immune cell responses and to induce tumor development respectively. The inventors then investigated whether anti-LILRB2 were capable to block these interactions in order to restore immune cell functions and prevent tumor growth. To study the inhibition of LILRB2/HLA-G interaction, the inventors first designed an ELISA assay to evaluate the blocking capacity of the Nbs. For this purpose, rhLILRB2-Fc proteins were coated on microtiter plate before being co-incubated with HLA-G6 protein in presence or not of the Nbs. It has been demonstrated that rh-LILRB2-Fc receptors have a strong affinity for the soluble HLA-G6 isoform. As shown on FIG. 6, isotype control monoclonal antibody interferes with the HLA-G6/LILRB2 interaction (24% of blocking) as well as the H-300 polyclonal antibody which was not reported to be blocking (26% of blocking). However, the anti-LILRB2 monoclonal blocking antibody 27D6 strongly abrogated the interaction between HLA-G6 and LILRB2 receptors (100% of blocking). Regarding to the anti-LILRB2 Nbs, the inventors determined that 7 Nbs (A2, C7, C9, D8, E7, F5 and G10) weakly inhibit the interaction (<30%), 3 Nbs showed a partial inhibition: D12 (44.8%), G3 (39.4%) and H12 (50%), whereas the B8 Nb completely inhibits the HLA-G6/LILRB2 interaction (100% of blocking).

B8 Nb Partially Inhibits LILRB2/ANGPTL2 Interaction

It was demonstrated that LILRB2/ANGPTL2 interaction promotes tumor development. Indeed, interaction between LILRB2 receptors, expressed by cancer cells, and ANGPTL2 protein, autocrine expression, leads to tumor proliferation, inhibition of tumor apoptosis and differentiation of tumor cells. To determine if the anti-LILRB2 Nbs were able to block this interaction, the inventors set up an ELISA to evaluate the interaction between rhLILRB2-Fc and ANGPTL2 proteins. As previously described, rhLILRB2-Fc proteins were coated on microtiter plate before being co-incubated with rhANGPTL2 in presence or not of anti-LILRB2 antibodies or Nbs. Some Nbs partially blocked the LILRB2/ANGPTL2 interaction (<24% inhibition of binding) (data not shown). B8 Nb strongly blocked this interaction (51.4% inhibition of binding) in comparison to the control without Nb while A2, H12 and G10 showed a weak blocking (24%, 20% and 8% respectively) (FIG. 7).

1.-17. (canceled)

18. A single domain antibody (sdAb) that specifically binds to Leukocyte immunoglobulin-like receptor subfamily B member 2 (LILRB2).

19. The sdAb of claim 18, wherein the LILRB2 is human LILRB2.

20. The sdAb of claim 18, wherein the sdAb does not bind to Leukocyte immunoglobulin-like receptor subfamily B member 1 (LILRB1).

21. The sdAb of claim 20, wherein the LILRB1 is human LILRB1.

22. The sdAb of claim 18, comprising at least one complementarity determining region (CDR), wherein the CDR comprises a sequence set forth in SEQ ID NO: 3, 6, 9, 12, 15, 18, 21, 24, 27, 30 or 33, or an amino acid sequence that differs from SEQ ID NO: 3, 6, 9, 12, 15, 18, 21, 24, 27, 30 or 33 by one, two, or three amino acid modifications.

23. The sdAb of claim 18, comprising:
(a) a CDR1 comprising the sequence of SEQ ID NO:1 or an amino acid sequence that differs from SEQ ID NO:1 by one, two, or three amino acid modifications,
a CDR2 comprising the sequence of SEQ ID NO:2 or an amino acid sequence that differs from SEQ ID NO:2 by one, two, or three amino acid modifications, and
a CDR3 comprising the sequence of SEQ ID NO:3 or an amino acid sequence that differs from SEQ ID NO:3 by one, two, three or four amino acid modifications;
(b) a CDR1 comprising the sequence of SEQ ID NO:4 or an amino acid sequence that differs from SEQ ID NO:4 by one, two, or three amino acid modifications,
a CDR2 comprising the sequence of SEQ ID NO:5 or an amino acid sequence that differs from SEQ ID NO:5 by one, two, or three amino acid modifications, and
a CDR3 comprising the sequence of SEQ ID NO:6 or an amino acid sequence that differs from SEQ ID NO:6 by one, two, three or four amino acid modifications;
(c) a CDR1 comprising the sequence of SEQ ID NO:7 or an amino acid sequence that differs from SEQ ID NO:7 by one, two, or three amino acid modifications,
a CDR2 comprising the sequence of SEQ ID NO:8 or an amino acid sequence that differs from SEQ ID NO:8 by one, two, or three amino acid modifications, and
a CDR3 comprising the sequence of SEQ ID NO:9 or an amino acid sequence that differs from SEQ ID NO:9 by one, two, three or four amino acid modifications;
(d) a CDR1 comprising the sequence of SEQ ID NO:10 or an amino acid sequence that differs from SEQ ID NO:10 by one, two, or three amino acid modifications,
a CDR2 comprising the sequence of SEQ ID NO:11 or an amino acid sequence that differs from SEQ ID NO:11 by one, two, or three amino acid modifications, and
a CDR3 comprising the sequence of SEQ ID NO:12 or an amino acid sequence that differs from SEQ ID NO:12 by one, two, three or four amino acid modifications;
(e) a CDR1 comprises comprising the sequence of SEQ ID NO:13 or an amino acid sequence that differs from SEQ ID NO:13 by one, two, or three amino acid modifications,
a CDR2 comprising the sequence of SEQ ID NO:14 or an amino acid sequence that differs from SEQ ID NO:14 by one, two, or three amino acid modifications, and
a CDR3 comprising the sequence of SEQ ID NO:15 or an amino acid sequence that differs from SEQ ID NO:15 by one, two, three or four amino acid modifications;
(f) a CDR1 comprising the sequence of SEQ ID NO:16 or an amino acid sequence that differs from SEQ ID NO:16 by one, two, or three amino acid modifications,
a CDR2 comprising the sequence of SEQ ID NO:17 or an amino acid sequence that differs from SEQ ID NO:17 by one, two, or three amino acid modifications, and
a CDR3 comprising the sequence of SEQ ID NO:18 or an amino acid sequence that differs from SEQ ID NO:18 by one, two, three or four amino acid modifications;
(g) a CDR1 comprising the sequence of SEQ ID NO:19 or an amino acid sequence that differs from SEQ ID NO:19 by one, two, or three amino acid modifications,
a CDR2 comprising the sequence of SEQ ID NO:20 or an amino acid sequence that differs from SEQ ID NO:20 by one, two, or three amino acid modifications, and
a CDR3 comprising the sequence of SEQ ID NO:21 or an amino acid sequence that differs from SEQ ID NO:21 by one, two, three or four amino acid modifications;
(h) a CDR1 comprising the sequence of SEQ ID NO:22 or an amino acid sequence that differs from SEQ ID NO:22 by one, two, or three amino acid modifications,
a CDR2 comprising the sequence of SEQ ID NO:23 or an amino acid sequence that differs from SEQ ID NO:23 by one, two, or three amino acid modifications, and
a CDR3 comprising the sequence of SEQ ID NO:24 or an amino acid sequence that differs from SEQ ID NO:24 by one, two, three or four amino acid modifications;
(i) a CDR1 comprising the sequence of SEQ ID NO:25 or an amino acid sequence that differs from SEQ ID NO:25 by one, two, or three amino acid modifications,
a CDR2 comprising the sequence of SEQ ID NO:26 or an amino acid sequence that differs from SEQ ID NO:26 by one, two, or three amino acid modifications, and
a CDR3 comprising the sequence of SEQ ID NO:27 or an amino acid sequence that differs from SEQ ID NO:27 by one, two, three or four amino acid modifications;
(j) a CDR1 comprising the sequence of SEQ ID NO:28 or an amino acid sequence that differs from SEQ ID NO:28 by one, two, or three amino acid modifications,
a CDR2 comprising the sequence of SEQ ID NO:29 or an amino acid sequence that differs from SEQ ID NO:29 by one, two, or three amino acid modifications, and
a CDR3 comprising the sequence of SEQ ID NO:30 or an amino acid sequence that differs from SEQ ID NO:30 by one, two, three or four amino acid modifications; or
(k) a CDR1 comprising the sequence of SEQ ID NO:31 or an amino acid sequence that differs from SEQ ID NO:31 by one, two, or three amino acid modifications,
a CDR2 comprising the sequence of SEQ ID NO:32 or an amino acid sequence that differs from SEQ ID NO: 32 by one, two, or three amino acid modifications, and
a CDR3 comprising the sequence of SEQ ID NO:33 or an amino acid sequence that differs from SEQ ID NO:33 by one, two, three or four amino acid modifications.

24. The sdAb of claim 18, comprising a CDR1 comprising the sequence of SEQ ID NO:1, a CDR2 comprising the sequence of SEQ ID NO:2, and a CDR3 comprising the sequence of SEQ ID NO:3.

25. The sdAb of claim 18, comprising the sequence of any one of SEQ ID NO: 34 to SEQ ID NO: 44 or a sequence having at least 80%, 90%, 92%, 94%, 95%, 96%, 97%, 98%, 99% or more amino acid sequence identity thereto.

26. The sdAb of claim 18, comprising the sequence of SEQ ID NO: 34.

27. The sdAb of claim 18, wherein the sdAb inhibits the interaction between LTLRB2 and human leukocyte antigen-G (HLA-G).

28. The sdAb of claim 18, wherein the sdAb inhibits the interaction between LTLRB2 and Angiopoietin Like 2 (ANGPTL2).

29. An isolated nucleic acid comprising a sequence encoding the sdAb of claim 18.

30. The isolated nucleic acid of claim 29, wherein the sequence is selected from the group consisting of SEQ ID NOs: 45-55.

31. A vector comprising the isolated nucleic acid of claim 29.

32. A chimeric antigen receptor (CAR) comprising the sdAb of claim 18.

33. A cell comprising the isolated nucleic acid of claim 29.

34. A cell expressing the CAR of claim 32

35. The cell of claim 34, wherein the cell is a T cell, a CD4+ T cell, a CD8+ T cell, a B cell, a NK cell, a NKT cell, a monocyte, or a dendritic cell.

36. A pharmaceutical composition comprising the sdAb of claim 18, and a pharmaceutically acceptable carrier.

37. A pharmaceutical composition comprising the cell of claim 34, and a pharmaceutically acceptable carrier.

38. A method for treating a cancer in a subject in need thereof, comprising administering to the subject the pharmaceutical composition of claim 36,

39. The method of claim 38, wherein the cancer overexpresses LILBR2.

40. The method of claim 38, wherein the cancer is lung cancer, non-small cell lung cancer (NSCLC), pancreatic cancer, pancreatic ductal carcinoma, Chronic Lymphocytic Leukemia (CLL), Acute Myeloid Leukemia (AML), endometrial cancer, hepatocellular carcinoma, melanoma, ovarian cancer, breast cancer, colorectal cancer, glioma, stomach cancer, renal cancer, testis cancer, Esophageal cancer, Cervical cancer, Lewis Lung cancer of mice, Leukemia, Thyroid cancer, Liver cancer, Urothelial cancer or Head and neck cancer.

41. An in vitro or ex vivo method for detecting LILRB2 on tumoral cells or tissues, comprising contacting the tumoral cells or tissued with the sdAb of claim 18.


SEQUENCE LISTING (55 sequences)

SEQ ID NO: 1 - protein, 8 aa, Artificial Sequence
  ILT4-B8 CDR1
GFTLDYYA

SEQ ID NO: 2 - protein, 8 aa, Artificial Sequence
  ILT4-B8 CDR2
IGNSGDST

SEQ ID NO: 3 - protein, 27 aa, Artificial Sequence
  ILT4-B8 CDR3
AARKGFASSCHGLGAAYDSDYESLYDY

SEQ ID NO: 4 - protein, 8 aa, Artificial Sequence
  ILT4-C7 CDR1
GFALEHYS

SEQ ID NO: 5 - protein, 8 aa, Artificial Sequence
  ILT4-C7 CDR2
ISNSGHTT

SEQ ID NO: 6 - protein, 16 aa, Artificial Sequence
  ILT4-C7 CDR3
AATPRGWGLTSNQYEY

SEQ ID NO: 7 - protein, 8 aa, Artificial Sequence
  ILT4-C9 CDR1
GRTLNGYT

SEQ ID NO: 8 - protein, 8 aa, Artificial Sequence
  ILT4-C9 CDR2
IRAIDGST

SEQ ID NO: 9 - protein, 19 aa, Artificial Sequence
  ILT4-C9 CDR3
AARLRVSNGNAWSSSSYLH

SEQ ID NO: 10 - protein, 8 aa, Artificial Sequence
  ILT4-A2 CDR1
GFNVNAYH

SEQ ID NO: 11 - protein, 7 aa, Artificial Sequence
  ILT4-A2 CDR2
ISSDHST

SEQ ID NO: 12 - protein, 16 aa, Artificial Sequence
  ILT4-A2 CDR3
AATANQLWQVWMGITS

SEQ ID NO: 13 - protein, 8 aa, Artificial Sequence
  ILT4-D8 CDR1
GLTSRPYY

SEQ ID NO: 14 - protein, 8 aa, Artificial Sequence
  ILT4-D8 CDR2
ISSREGST

SEQ ID NO: 15 - protein, 16 aa, Artificial Sequence
  ILT4-D8 CDR3
AGRYSTCSGMARDYNY

SEQ ID NO: 16 - protein, 8 aa, Artificial Sequence
  ILT4-D12 CDR1
GFTLDHYA

SEQ ID NO: 17 - protein, 8 aa, Artificial Sequence
  ILT4-D12 CDR2
INVNRGTS

SEQ ID NO: 18 - protein, 23 aa, Artificial Sequence
  ILT4-D12 CDR3
AALLEGASGDVCDDVNQNYGMDY

SEQ ID NO: 19 - protein, 8 aa, Artificial Sequence
  ILT4-E7 CDR1
GSTSRHYY

SEQ ID NO: 20 - protein, 8 aa, Artificial Sequence
  ILT4-E7 CDR2
ITSREGST

SEQ ID NO: 21 - protein, 16 aa, Artificial Sequence
  ILT4-E7 CDR3
AARYSSCSGMPSDYNY

SEQ ID NO: 22 - protein, 8 aa, Artificial Sequence
  ILT4-F5 CDR1
GFNVNAYH

SEQ ID NO: 23 - protein, 7 aa, Artificial Sequence
  ILT4-F5 CDR2
ISSDRET

SEQ ID NO: 24 - protein, 16 aa, Artificial Sequence
  ILT4-F5 CDR3
AATANQLWRLWMGISS

SEQ ID NO: 25 - protein, 8 aa, Artificial Sequence
  ILT4-G3 CDR1
GFTSRHYY

SEQ ID NO: 26 - protein, 8 aa, Artificial Sequence
  ILT4-G3 CDR2
ISSREGST

SEQ ID NO: 27 - protein, 16 aa, Artificial Sequence
  ILT4-G3 CDR3
AGRYSSCSGMPRDYNY

SEQ ID NO: 28 - protein, 8 aa, Artificial Sequence
  ILT4-G10 CDR1
GFTFFAYT

SEQ ID NO: 29 - protein, 8 aa, Artificial Sequence
  ILT4-G10 CDR2
IDWTGSSS

SEQ ID NO: 30 - protein, 20 aa, Artificial Sequence
  ILT4-G10 CDR3
ATLCSLLLRVARPIIADGGV

SEQ ID NO: 31 - protein, 8 aa, Artificial Sequence
  ILT4-H12 CDR1
GFTLDSYA

SEQ ID NO: 32 - protein, 8 aa, Artificial Sequence
  ILT4-H12 CDR2
ISSSGGST

SEQ ID NO: 33 - protein, 21 aa, Artificial Sequence
  ILT4-H12 CDR3
AALREGSYYPDDDACRDGMDY

SEQ ID NO: 34 - protein, 134 aa, Artificial Sequence
  ILT4-B8
EVQLVESGGGLVQPGGSLRLSCVASGFTLDYYAISWFRRAPGKEREGVSCIGNSGDSTNYADSVKGRFTISRDNAKNTVYLQMNSLKPEETAVYYCAARKGFASSCHGLGAAYDSDYESLYDYWGQGTQVTVSS

SEQ ID NO: 35 - protein, 125 aa, Artificial Sequence
  ILT4-C7
MAQVQLVESGGGLVQPGGSLRLSCAASGFALEHYSIGWFRQAPGKEREGVSCISNSGHTTKYADSVKGRFTVSRDNAKNTVYLQMNSLKPEDTAVYYCAATPRGWGLTSNQYEYWGQGTQVTVSS

SEQ ID NO: 36 - protein, 126 aa, Artificial Sequence
  ILT4-C9
EVQLVESGGGLVQAGGSLRLSCAASGRTLNGYTTGWFRQAPGKEREFVATIRAIDGSTSYADSVMGRFTISRDNAKNTLYLEMNRLKPEDSALYYCAARLRVSNGNAWSSSSYLHWGRGTQVTVSS

SEQ ID NO: 37 - protein, 120 aa, Artificial Sequence
  ILT4-A2
QLQLVESGGGLVQPGGSLRLSCAASGFNVNAYHIGWFRQAPGKEREGVSFISSDHSTNYADSVKGRFTISRDSGTVYLQMNRLKPEDTATYYCAATANQLWQVWMGITSWGQGTQVTVSS

SEQ ID NO: 38 - protein, 123 aa, Artificial Sequence
  ILT4-D8
QLQLVESGGGLVQPGGSLRLSCAASGLTSRPYYIGWFRQSPGKEREGVSCISSREGSTNYADSVKDRFTISRDNAQSMFYLQMNSLKSEDTAVYYCAGRYSTCSGMARDYNYWGQGTQVTVSS

SEQ ID NO: 39 - protein, 130 aa, Artificial Sequence
  ILT4-D12
EVQLVESGGGLVQPGGSLKLSCAASGFTLDHYAIGWFRQAPGEKREGVSCINVNRGTSAYADSVKGRFTISRDNAKNTVYLQMDNLRPEDTAVYYCAALLEGASGDVCDDVNQNYGMDYWGKGTLVTVSS

SEQ ID NO: 40 - protein, 123 aa, Artificial Sequence
  ILT4-E7
EVQLVESGGGLVQPGGSLRLSCAASGSTSRHYYIGWFRQAPGKEREGVSCITSREGSTNYADSVKDRFTISRDNAQNMFYLQMNSLKSEDTAVYYCAARYSSCSGMPSDYNYWGRGTQVTVSS

SEQ ID NO: 41 - protein, 122 aa, Artificial Sequence
  ILT4-F5
EVQLVESGGGLVQPGGSLRLSCAASGFNVNAYHIGWFRQAPGEEREGVSFISSDRETNYADSVKGRFTISRDSAKNTVYLQMNRLKPEDTAVYYCAATANQLWRLWMGISSWGQGTQVTVSS

SEQ ID NO: 42 - protein, 123 aa, Artificial Sequence
  ILT4-G3
EVQLVESGGGLVQPGGSLRLSCAASGFTSRHYYIGWFRQSPGKEREGLSCISSREGSTNYADSVQDRFTISRDNTQNMFYLQMNSLKSEDTAVYYCAGRYSSCSGMPRDYNYWGRGTQVTVSS

SEQ ID NO: 43 - protein, 127 aa, Artificial Sequence
  ILT4-G10
QVQLVESGGGLVHPGGSLRLSCAASGFTFFAYTMGWFRQAPGKEREVLSGIDWTGSSSNYADSVRGRFTISRHNAKNTLYLQMNSLKPDDTAVYYCATLCSLLLRVARPIIADGGVWSQVTQVTVSS

SEQ ID NO: 44 - protein, 128 aa, Artificial Sequence
  ILT4-H12
EVQLVESGGGLVQPGGSLRLSCAASGFTLDSYAVGWFRQAPGKEREGVSCISSSGGSTHYADSVKGRFTISRDNAKNTVYLQMSSLKPEDTASYYCAALREGSYYPDDDACRDGMDYWGKGTQVTVSS

SEQ ID NO: 45 - DNA, 402 bp, Artificial Sequence
  ILT4-B8
gaggtgcagctggttgagtcggggggaggtttggtgcagcctggggggtctctgagactctcctgtgtagcctctggattcactttggattattatgccataagctggttccgccgggccccagggaaggagcgtgagggtgtctcatgtattggtaatagtggtgatagcacaaactatgcagactccgtgaagggccgattcaccatctccagagacaacgccaagaacacggtttatctgcaaatgaacagcctgaaacctgaggaaacagccgtttattactgcgcagcgcgaaaagggttcgctagttcctgtcacggcctcggggctgcatacgatagtgactatgaatcgttgtatgactactggggccaggggacccaggtcaccgtctcctca

SEQ ID NO: 46 - DNA, 370 bp, Artificial Sequence
  ILT4-C7
tcaggtgcagctcgtggagtcggggggaggcttggtgcagcctggggggtctctgagactctcctgtgcagcctctggattcgctttggaacattattccataggctggttccgccaggccccagggaaggagcgtgagggggtctcatgtattagtaatagtgggcataccacaaagtatgcagactccgtgaagggccgattcaccgtctccagagacaacgccaagaacacggtgtatctgcaaatgaacagcctgaagcctgaggacacagccgtttattactgtgcagcgacaccaaggggttggggcctaacgtcgaatcagtatgaatactggggccaggggacccaggtcaccgtctcatca

SEQ ID NO: 47 - DNA, 378 bp, Artificial Sequence
  ILT4-C9
gaggtgcagctggtagagtctgggggaggattggtgcaggctgggggctctctgagactctcctgtgcagcctctggacgcaccctcaatggttataccactggctggttccgccaggctccagggaaggagcgcgagtttgtagccactattcgcgcgattgatggtagcacatcctatgcagactccgtgatgggccgattcaccatctccagagacaacgccaaaaatacgctctatcttgaaatgaacagactgaaacctgaggactcggccctgtattattgtgcggcgcggctcagagtgagcaatggtaacgcatggtcatcatctagctatctccactggggccgggggacccaggtcaccgtctcctca

SEQ ID NO: 48 - DNA, 361 bp, Artificial Sequence
  ILT4-A2
tcagttgcagctcgtggagtctgggggaggcttggtgcagcctggggggtctctgagactctcctgtgcagcctctggattcaacgtgaatgcttatcacataggctggttccgccaggccccaggaaaggagcgtgagggggtctcattcattagtagtgatcatagcacaaactatgcagactccgtgaagggacgattcaccatctccagagacagcggtacggtgtatctgcaaatgaacagactgaaacctgaggacacagccacttactattgtgcagcaaccgccaatcaactgtggcaagtatggatgggcattacgtcctggggccaggggacccaggtcaccgtctcctcg

SEQ ID NO: 49 - DNA, 370 bp, Artificial Sequence
  ILT4-D8
tcagttgcagctcgtggagtcggggggaggcttggtgcagcctggggggtctctgagactctcctgtgcagcctctggattgacttcgcgcccttattacataggttggtttcgccagtccccagggaaggagcgtgagggagtctcatgtattagtagtagggaaggtagcacaaactacgcagactccgtgaaggatcgattcaccatctccagagataacgctcagagtatgttttatctgcaaatgaacagcctgaaatctgaggacacagccgtttattactgtgcaggacgttattcgacatgttcaggaatggcccgcgattataactactggggtcaggggacccaggtcaccgtctcctca

SEQ ID NO: 50 - DNA, 390 bp, Artificial Sequence
  ILT4-D12
gaggtgcagctggtagagtctgggggaggcttggtgcagcctggggggtctctgaaactctcctgtgcagcctctggattcactttggatcattatgccataggctggttccgccaggccccaggcgaaaagcgtgaaggcgtctcatgtattaatgttaatcgtggtacctccgcctatgcagactccgtgaagggccgtttcaccatctcgcgagacaacgccaagaacacggtgtatctgcaaatggacaacctgagacctgaggacacagccgtttattactgtgcagccctactcgagggggcctcaggcgatgtgtgtgatgacgtcaatcaaaactacggcatggactactggggcaaagggaccctggtcaccgtctcctca

SEQ ID NO: 51 - DNA, 369 bp, Artificial Sequence
  ILT4-E7
gaggtgcagctggttgagtcggggggaggcttggtgcagcctggggggtctctgagactctcctgtgcagcctctggatcgacttcgcgccattattacataggctggttccgccaggccccaggaaaggagcgtgaaggtgtctcatgtattactagtagggaaggtagcacaaactacgcagactccgtgaaggaccgattcaccatctccagagacaacgctcagaatatgttttatttgcaaatgaacagcctgaaatctgaggacacagccgtttattactgtgcagcacgttattcgagttgttcaggaatgccctcagattataactactggggtcgggggacccaggtcaccgtctcctca

SEQ ID NO: 52 - DNA, 366 bp, Artificial Sequence
  ILT4-F5
gaggtgcagctggtagagtcggggggaggcttggtgcagcctggggggtctctgagactctcctgtgcagcctctggattcaacgtgaatgcttatcacataggctggttccgccaggccccaggagaggagcgtgaaggggtctcattcattagtagtgatcgtgagacaaactatgcagactccgtgaagggccgattcaccatctccagagacagcgccaagaacacggtgtatctgcaaatgaacagactgaaacctgaggacacagccgtttattactgtgcagcaaccgccaatcaactgtggcgactatggatgggcattagttcctggggccaggggacccaggtcaccgtctcctca

SEQ ID NO: 53 - DNA, 369 bp, Artificial Sequence
  ILT4-G3
gaggtgcagctggtagagtctgggggaggcttggtgcagcctggggggtctctgagactctcctgtgcagcctctggattcacttcgcgccattattacataggttggtttcgccagtccccagggaaggagcgtgaggggctctcatgtattagtagtagggaaggtagcacaaactacgcagactccgtacaggaccgattcaccatctccagagacaacactcagaatatgttttatttgcaaatgaacagcctgaaatctgaggacacagccgtttattactgtgcaggacgttattcgagttgttcaggaatgccccgggattataactactggggtcgggggacccaggtcaccgtctcctca

SEQ ID NO: 54 - DNA, 381 bp, Artificial Sequence
  ILT4-G10
caggtgcagctcgtggagtccgggggaggcttggtgcaccctggggggtctctgagactctcctgtgcagcctctggattcactttttttgcgtataccatgggctggttccgccaggctccagggaaggagcgtgaggttctctcaggtattgactggaccgggagtagctcaaactatgcagactccgtgaggggccgattcaccatctccagacacaacgccaagaatacgctgtatctacaaatgaacagcctgaaacctgacgacacggccgtgtattactgtgcgacactttgttctctactgttacgggtcgcgcggccaattattgctgatgggggagtttggagccaggtgacccaggtcaccgtctcctca

SEQ ID NO: 55 - DNA, 384 bp, Artificial Sequence
  ILT4-H12
gaggtgcagctggtagagtctgggggaggcttggtgcagcctggggggtctctgagactctcctgtgcagcctctggattcactttggattcatatgccgtaggctggttccgccaggccccagggaaggagcgtgagggagtctcatgtattagtagtagtggtggtagcacacactatgcagactccgtgaagggccgattcaccatctccagagacaacgccaagaacacggtgtatctgcaaatgagcagcctgaaacctgaggacacagccagttattactgtgcagcacttcgcgaaggttcttactatcccgacgatgacgcgtgtcgtgacggcatggactactggggcaaagggacccaggtcaccgtctcctca